(19) United States
(12) Patent Application Publication
(10) Pub. No.: US 2014/0179726 A1
(43) Pub. Date: Jun. 26, 2014

(54) GUT MICROFLORA AS BIOMARKERS FOR THE PROGNOSIS OF CIRRHOSIS AND BRAIN DYSFUNCTION

(71) Applicants: Jasmohan Bajaj, Richmond, VA (US); Arun Sanyal, Mechanicsville, VA (US); Patrick M. Gillevet, Oakton, VA (US)

(72) Inventors: Jasmohan Bajaj, Richmond, VA (US); Arun Sanyal, Mechanicsville, VA (US); Patrick M. Gillevet, Oakton, VA (US)

(73) Assignees: VIRGINIA COMMONWEALTH UNIVERSITY, Richmond, VA (US); GEORGE MASON UNIVERSITY, Fairfax, VA (US); THE U.S DEPARTMENT OF VETRANS AFFAIRS, Washington, DC (US)

(21) Appl. No.: 14/118,566

(22) PCT Filed: May 18, 2012
(86) PCT No.: PCT/US2012/038555

§ 371 Date: Feb. 17, 2014

Related U.S. Application Data
(60) Provisional application No. 61/621,767, filed on Apr. 9, 2012.
(60) Provisional application No. 61/488,019, filed on May 19, 2011.

(51) Int. Cl. C12Q 1/68; A61K 31/437

(52) U.S. Cl. CPC C12Q 1/689; A61K 31/437
USPC 514/279; 435/6.12

(57) ABSTRACT

A systems biology approach is used to characterize and relate the intestinal (gut) microbiome of a host organism (e.g. a human) to physiological processes within the host. Information regarding the types and relative amounts of gut microflora is correlated with physiological processes indicative of e.g., a patient's risk of developing a disease or condition, likelihood of responding to a particular treatment, for adjusting treatment protocols, etc. The information is also used to identify novel suitable therapeutic targets and/or to develop and monitor the outcome of therapeutic treatments. An exemplary disease/condition is the development of hepatic encephalopathy (HE), particularly in patients with liver cirrhosis.

### BACKGROUND OF THE INVENTION

1. Field of the Invention

The invention generally relates to methods for predicting, for patients, a level of risk for developing a disease or condition associated with particular patterns of gut microflora (microbiome) colonization. In particular, the invention provides methods of correlating the presence or absence and/or relative abundances of gut microflora with a patient's risk of developing an associated disease or condition, and developing suitable treatments based on the correlation.

2. Background of the Invention

The human body, consisting of about 100 trillion cells, carries about ten times as many microorganisms in the intestines. It is estimated that these gut flora have around 100 times as many genes in aggregate as there are in the human genome. Research suggests that the relationship between gut flora and humans is not merely commensal (a non-harmful coexistence), but rather a symbiotic relationship. These microorganisms perform a host of useful functions, such as fermenting unused energy substrates, training the immune system, forming a protective mucosal biofilm, preventing growth of harmful, pathogenic bacteria, regulating the development of the gut, producing vitamins for the host (e.g. biotin and vitamin K), producing hormones to direct the host to store fats, producing signaling molecules that promote homeostasis, metabolizing drugs and xenobiotics, etc. However, in certain conditions, some species are thought to be capable of causing or promoting disease.

For example, cirrhosis is often complicated by hepatic encephalopathy (HE), a condition characterized by cognitive impairment and poor survival, and there is evidence that pathogenic abnormalities in HE are related to the gut flora and their by-products, such as ammonia and endotoxin in the setting of intestinal barrier dysfunction and systemic inflammation. However, no clear correspondence between cognitive impairment and gut microflora has been established.

The current treatments for HE rely on manipulation of the gut flora. However, this treatment is not successful in all cases. Success is hampered by a poor understanding of the identity and mechanism of action of gut flora.

Moreover, prior techniques for the characterization of gut flora has been severely limited by the use of culture-based techniques that do not support the growth of the majority of the intestinal bacteria.

The prior art has thus far failed to provide methods of readily and accurately assessing the complement of microflora present in an individual, and/or of correlating the presence of particular microbes with particular diseases and conditions, and/or the risk of developing the same. This is particularly true with respect to patients with HE.

### SUMMARY OF THE INVENTION

The invention provides methods for assessing the gut microflora of individuals, for identifying appropriate therapeutic targets and developing appropriate treatment protocols based on the assessment, and for monitoring the progress or outcome of treatment strategies. The methods involve the use of a systems biology approach using correlation network analysis (or similar approaches including without limitation non-parametric multivariate analysis, a Support Vector Machine, correlation difference network analysis, Dirichlet models, Bayesian models, and Linear models) to characterize the intestinal microflora of an individual, and to relate the patterns or distributions of microflora (“signatures”) to physiological processes, metabolic processes (metabolome), and clinical measures of health. The complex interactions of the microbiome and the human host are defined herein as the metabiome. For example, the signatures are correlated with various hallmarks or symptoms of disease and the activation and/or deactivation of physiological processes related to disease, based on known, previously established prototype signatures. Information gained by the methods of the invention may be advantageously used, for example, to diagnose conditions, to confirm diagnoses, to predict a patient's risk of developing a disease or condition (e.g. prior to the onset of symptoms), to identify suitable therapeutic targets, and to monitor or track the outcome of therapeutic intervention. In particular, methods related to individuals who suffer from liver diseases, as well as those who have HE or who are at risk for developing HE are provided.

The present invention provides methods of assessing the presence or the risk of development of encephalopathy in a patient with liver disease. The methods comprise the steps of 1) analyzing gut microflora of said patient in order to determine a gut microbiome signature for said patient; 2) comparing said gut microbiome signature of said patient to one or more gut microbiome reference signatures, wherein said one or more gut microbiome reference signatures include at least one of a positive gut microbiome reference signature based on results from control subjects with encephalopathy and a negative gut microbiome reference signature based on results from control subjects without encephalopathy; and if said gut microbiome signature for said patient statistically significantly matches said positive gut microbiome reference signature, (e.g. includes the same types and/or the same relative abundances, ratios, etc. of microflora in statistically significant amounts), then concluding that said patient has or is at risk of developing encephalopathy; and/or if said gut microbiome signature for said patient statistically significantly matches said negative gut microbiome reference signature, then concluding that said patient does not have or is not at risk of developing encephalopathy. In some embodiments, a statistically significant match has a P value of 0.05 or less. In some embodiments, the gut microflora is analyzed in a biological sample preferably selected from a stool sample, a sample of the lumen content, a mucosal biopsy sample, an oral sample, a blood sample and a urine sample. In other embodiments, the gut microbiome signature may include one or more of: bacterial taxa identified in said biological sample; bacterial metabolic products in said biological sample; and proteins in said biological sample. In yet other embodiments, the gut microbiome signature is based on an analysis of amplification products of DNA and/or RNA of said gut microflora, e.g. is based on an analysis of amplification products of genes coding for one or more of: Small Subunit rRNA, Intervening Transcribed Spacer, and Large Subunit rRNA. In some embodiments, the gut microbiome signature includes results obtained by assaying the mRNA composition of said biological samples. In some embodiments, the liver disease is cirrhosis and the encephalopathy is hepatic encephalopathy (HE). In some embodiments of the invention, the gut microbiome signature of said patient includes an indication of the presence and/or relevant abundance of at least one of AI caligeneceae, Blautia, Burkholderia, Enterobacteriaceae, Fecalibacterium, Fusobacteriaceae, Incertae Sedis XIV, Lachnospiraceae, Porphyromonadaceae, Roseburia, Rwninococcaceae and Veillonellaceae. In other embodiments, when the gut microflora signature of said patient indicates the presence of Alcaligeneceae and Porphyromanadaceae in said gut microflora, then said concluding step results in a conclusion that said patient has or is at risk of developing encephalopathy. In other embodiments, the method further comprises the step of assessing, based on said gut microbiome signature, the presence or the risk of development of inflammation, endotoxemia, and/or endothelial dysfunction in said patient. In yet other embodiments, the one or more symptoms of a disease or condition is differentiated from normal conditions using at least one methodology selected from the group consisting of non-parametric multivariate analysis, a Support Vector Machine, correlation network analysis, correlation difference network analysis, Dirichlet models, Bayesian models, and Linear models.

The invention also provides a treatment method for a patient with a liver disease. The method comprises the steps of 1) analyzing gut microflora of said patient in order to determine a gut microbiome signature for said patient; 2) comparing said gut microbiome signature of said patient to one or more gut microbiome reference signatures; and, based on said step of comparing, 3) concluding whether or not said patient has or is at risk for developing at least one of one or more conditions of interest; and if said patient has or is at risk for developing at least one of said one or more conditions of interest, then selecting from one or more treatment protocols appropriate for said one or more conditions of interest. In some embodiments, the one or more conditions of interest include encephalopathy, inflammation, endotoxemia, endothelial dysfunction and coma. In other embodiments, the treatment protocols include one or more of: anti-viral therapy for hepatitis B, C and/or D; weight loss therapy; surgery for non-alcoholic liver disease and obesity-associated liver disease, alcohol abstinence for alcoholic liver disease, therapy for Wilson's disease, alpha-1 anti-trypsin repletion, and therapies specific for hepatic encephalopathy and liver transplant.

The invention provides a method of monitoring the efficacy of a treatment protocol in a patient with liver disease or a condition associated with liver disease, comprising the steps of 1) analyzing gut microflora of said patient in order to determine a gut microbiome signature for said patient; and 2) comparing said gut microbiome signature of said patient to one or more gut microbiome reference signatures, wherein said one or more gut microbiome reference signatures include at least one of a positive gut microbiome reference signature based on results from control subjects with encephalopathy and a negative gut microbiome reference signature based on results from control subjects without encephalopathy; wherein if said gut microbiome signature for said patient statistically significantly matches said positive gut microbiome reference signature, then concluding that said treatment protocol is not efficacious. Alternatively, the process could conclude that said treatment protocol is efficacious if said gut microbiome signature for said patient statistically significantly matches said negative gut microbiome reference signature. However, a treatment protocol may be deemed efficacious even if the treated patient's signature does not match that of a healthy (or asymptomatic) control, so long as the signature indicates a change away from the signature of a control group with encephalopathy, e.g. lowered amounts of non-beneficial bacteria (e.g. at least about 10% lower, or 20, 30, 40, 50, 60, 70, 80, 90 or even 100% decrease in the presence of at least one unwanted bacterium, and/or a corresponding increase in at least one beneficial or desirable bacterium). In some embodiments, the method further comprises the step of repeating said steps of said method at multiple spaced-apart time intervals, e.g. said method is carried out prior to commencement of said treatment protocol, during said treatment protocol and/or after cessation of said treatment protocol.

### BRIEF DESCRIPTION OF THE DRAWINGS

FIG. 1. Principal Coordinate Analysis of the Fecal Microbiome of Controls and Cirrhotic Patients. This graph shows the variation in fecal microbiome plotted on a principal coordinate analysis plot. Points that are closer to each other are similar with respect to their stool microbiota. The healthy controls represented by the black dots are clustered together while the cirrhotic patients represented by the gray dots are distant from the controls. This indicates a difference in the stool microbiome of healthy controls compared to cirrhotic patients.

FIG. 2A-B. Correlation Network Analysis of Cirrhotic Patients with and without Hepatic Encephalopathy. Only correlations with a coefficient >r=0.90 are displayed. Grey nodes indicate microbiome families; white nodes indicate cognitive tests and black nodes are serum inflammatory markers. A dashed line connecting nodes indicate positive correlation and a solid line indicates negative correlation >0.90. The p values for the correlations are displayed on or near the lines connecting the nodes. MELD: model for end-stage liver disease score, DST: digit symbol test, LDTe: line drawing test errors. A, Patients with HE (n=17) have a high number of significant correlations. There are significant positive correlations between IL-23 and several bacterial families. Prevotellaceae and Fusobacteriaceae are positively correlated with inflammation. Since a low score on DST and high one on LDTe indicate poor performance, Alcaligenaceae and Porphyromonadaceae were correlated with poor cognition. The p-values for all these correlations are less than the 4^{th} decimal place indicating a very high significance. B, Patients without HE have very few significant correlations (n=8). There was a significant negative correlation between MELD score and Ruminococcaceae and a positive correlation between Veillonellaceae and Porphyromonadaceae.

FIG. 3A-F. Correlation Network and Sub-networks of the mucosal microbiome of HE patients. A, correlation Network of the mucosal microbiome of HE patients. As can be seen, autochthonous genera belonging to the Ruminococcaceae, Lachnospiraceae and Incertae Sedis families are associated with good cognition, lower MELD, lower ammonia, and decreased inflammation. Sub-networks from this complex network are displayed in the figures B-F; B, sub-network of the HE mucosa microbiome showing the negative correlation of the autochthonous bacteria to MELD score and inflammation; C, sub-network of the HE mucosa microbiome showing the negative correlation of the inflammatory cytokines, particularly IL-17 with autochthonous bacteria and positive correlation with Lures (indicating worse cognition with increased inflammation), endothelial activation (sICAM-I), MELD score and non-autochthonous bacterial genera (Burkholderiaceae, Erysipelothricaceae; D, a high lure number indicates poor cognition. This sub-network of the HE mucosa microbiome shows that lures are negatively correlated with autochthonous bacterial genera (Roseburia and Dorea) while they are correlated positively with Burkholderiaceae and Incertae sedis XI and as expected with ammonia and inflammatory cytokines; E, a high number on NCT-B indicates poor performance. This sub-network of the HE mucosal microbiome shows a negative correlation i.e. good NCT-B performance with the abundances of Ruminococcaceae_{—}Fecalibacterium. This autochthonous genus has been associated with lower MELD score, lower inflammation (IL-17 and IL-10) and is positively correlated with other beneficial autochthonous bacteria; F, Megasphaera was significantly more abundant in HE; in this sub-network Megasphaera abundance is significantly correlated with sVCAM-1 (marker of endothelial activation) and with poor cognitive performance (a high score on SDT and LDTt indicates poor while a high score on DST indicates good cognitive performance). Connecting dashed lines indicate a significant negative while solid lines mean a significantly positive correlation. Nodes in gray are bacterial genera, double cross hatch are inflammatory cytokines, white are cognitive tests, black are clinical variables, heavy cross hatch are markers of endothelial activation and fine cross hatch are neuro-glial markers. A high score on DST (digit symbol) and Targets indicates good cognition while a high score on the rest of the cognitive tests indicates poor performance. SDT: serial dotting, LDTt: Line tracing test time and NCT-A/B: number connection test A/B.

FIG. 4A-D. Correlation network and sub-networks of the mucosal microbiome of patients without HE. Indicators and abbreviations are the same as in FIG. 3. A, Correlation network of the mucosal microbiome of patients without HE. Autochthonous genera belonging to the Ruminococcaceae, Lachnospiraceae and Incertae Sedis families are associated with good cognition, lower MELD, ammonia, and inflammation; B, this sub-network of patients without HE shows that bacteria genera belonging to autochthonous families (Ruminococcaceae and Lachnospiraceae) are positively correlated with each other while negatively correlated with potentially pathogenic Enterobacteriaceae and Propionibacterium; C, this sub-network of the no-HE mucosal microbiome again shows the positive correlation of the autochthonous bacteria with each other and a negative correlation with time required to complete NCT-A, which indicates good cognitive performance; D, a high score on targets and low score on lures indicates good cognitive performance. We again found a correlation between poor performance on lures and targets with genera belonging to Por phyromonadaceae and Alcaligenaceae.

FIG. 5A-B is a schematic diagram and flow chart of a system and method for performing the various embodiments of the invention.

### DETAILED DESCRIPTION

The pathogenesis of HE spans several metabolic processes, and a systems biology approach was used as described herein to identify novel functional correlations between HE and gut microflora. As such, HE provides an exemplary system for the application of the methods and systems of the invention. For example, the studies disclosed herein successfully demonstrated a link between the composition of the gut microbiome and cognition, inflammation, and endothelial dysfunction in cirrhotic patients with and without HE. The a priori hypothesis was that the gut microbiome composition (“signature”) would be correlated with cognition and inflammation in cirrhotic patients with HE and that this association or signature would be different from those who have never developed HE. This hypothesis was confirmed, and has led to the development of methods of assessing the propensity (risk, likelihood, etc.) of a patient to develop a disease known to be associated with a particular pattern of gut microflora, methods of identifying suitable therapeutic targets (and hence targeted treatment protocols), methods of developing treatment protocols, and methods of monitoring the progress of treatment. In addition, the gut microflora signature may be used as the basis for developing targeted molecules to counter the inflammation, bacterial end-products and microflora and/or to produce prebiotics/probiotics/modified bacteria (e.g. genetically modified bacteria) to replenish, in individuals in need thereof, abnormally low quantities of autochthonous bacteria associated with the gut of healthy or asymptomatic individuals, and to reduce the harmful bacteria associated with untoward or undesirable conditions such as inflammation and brain dysfunction.

The following definitions are used throughout:

Gut. The gut of an individual generally comprises, for example, the stomach (or stomachs, in ruminants), the colon, the small intestine, the large intestine, cecum, and the rectum. However, in some embodiments, other organs and/or cavities may be included in this category. In addition, regions of the gut may be subdivided, e.g. the right vs the left side of the colon may have different microflora populations due to the time required for digesting material to move through the colon, and changes in its composition with time. Synonyms include the gastrointestinal tract, or possibly the digestive system, although the latter is generally also understood to comprise the mouth, esophagus, etc.

Microflora refers to the collective bacteria and other microorganisms in an ecosystem of a host (e.g. an animal such as a human) or in a single part of the host's body, e.g. the gut. An equivalent term is “microbiota”.

Microbiome: the totality of microbes (bacteria, fungae, protists), their genetic elements (genomes) in a defined environment, e.g. within the gut of a host.

Metabolome: all the metabolic compounds in a defined environment, e.g. within the gut of a host.

Immunome: all the immune interactions within the host and between the host and microbiome in a defined environment, e.g. within the gut of a host.

Metabiome: all the interactions between the microbiome, the human host and environment in a defined environment, e.g. the microbiome, metabolome, and immunome.

Transcriptome: the mRNA composition of a sample.

Prebiotics are non-digestible food ingredients that stimulate the growth and/or activity of bacteria in the digestive system. Typically, prebiotics are carbohydrates (such as oligosaccharides), but the term may include non-carbohydrates. The most prevalent forms of prebiotics are nutritionally classed as soluble fiber. Exemplary prebiotics include but are not limited to various short-chain, long-chain, and “full-spectrum” polysaccharides such as oligofructose, inulin, polysaccharides with molecular link-lengths from 2-64 links per molecule [e.g. Oligofructose-Enriched Inulin (OEI)], galactooligosaccharides, and others. The term prebiotics may refer to commercial preparations of purified forms of these substances, and/or to natural sources, e.g. soybeans, inulin sources (such as Jerusalem artichoke, jicama, and chicory root), raw oats, unrefined wheat, unrefined barley, yacon, oligosaccharides from milk, etc.

Probiotics are live microorganisms thought to be beneficial to the host organism, examples of which include lactic acid bacteria (LAB), bifidobacteria, certain yeasts and bacilli, etc. Treatment with probiotics as described herein may be implemented by their consumption as part of fermented foods with specially added active live cultures (e.g. yogurt, soy yogurt, kefir, various cheeses, etc.) or as dietary supplements (e.g. tablets, powders, liquids, etc. which contain probiotic organisms), or in any other form.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

In one embodiment, the present invention provides methods for diagnosing patients at risk for developing a disease or condition correlated with the presence or absence of (and/or the relative distribution of) particular taxa of microbes in the gut, or in a particular component of or location within the gut. Such patients may have a higher than average or higher than normal chance of developing overt symptoms of the disease or condition, compared to individuals who have different gut microbes, or different amounts of microbes, or different relative amounts of microbes. Early identification of such a propensity allows early intervention, e.g. by altering the identity and/or the relative abundance of gut microflora associated with, and possibly causing, the disease/condition, so that development of the disease/condition may be avoided, or delayed, or the associated symptoms may be lessened.

In some embodiments, the patient may already exhibit overtly one or more symptoms of a disease of interest. But, by using the methods of the invention, it is possible to ascertain whether or not a likely cause of the disease symptom(s) is gut microflora identity (composition of the microbiome) and/or distribution, and hence whether or not gut microflora are a likely target for successful treatment. In other embodiments, a subject may be asymptomatic with respect to a disease or condition of interest, but for some reason, may be deemed susceptible to developing the disease or condition, and the methods of the invention provide a way to predict whether or not this is likely to occur. In some embodiments, the identification of particular microflora (e.g. of particular phyla, genera or species of microbe) may allow targeted therapies directed against the microbe or microbes which are undesirable, and/or therapies which increase the amount of desirable gut microflora, e.g. those which compete with the undesirable microbes, and/or which supply activities or produce substances which are beneficial, especially with respect to the disease or condition of interest.

The methods of the invention may involve steps of identifying a patient, the health or medical condition of whom might benefit from the knowledge provided by the method. The patient may be completely asymptomatic at the time of the analysis (but for some reason, a medical professional determines that the patient may benefit from the practice of the invention, e.g. the patient may be known to have a liver condition or disease), or the patient may be in the early, or even later, stages of the disease, and can benefit from the knowledge of the status of the gut microflora. In order to practice the methods of the invention, generally a sample of gut microflora is obtained from the patient by any method known to those of skill in the art, and the sample is tested for the presence or absence of, and/or for the relative abundance of, at least one taxon of microbes. Generally, the taxa which are targeted for assessment are one or more taxa, the presence of which is known to be correlated with a particular disease or condition, or with particular symptoms associated or correlated with a disease/condition. In some embodiments, identification of a single or a few (e.g. about 10 or fewer, or about 100 or fewer) key microbes may be sufficient to link the presence of the microbes to the likely development of a disease. However, in other embodiments, a broad taxonomy determination is made, e.g. dozens, hundreds, or thousands (or more) taxa may be targeted for assessment of their presence and/or absence and/or relative abundance.

Suitable biological samples for interrogation using the methods of the invention include but are not limited to: samples of gut contents and/or mucosal biopsies obtained directly by an invasive technique e.g. by surgery, by rectal or intestinal sampling via colonoscopy-type procedures, or by other means. Preferably, samples are obtained by less invasive methods, e.g. stool samples, including stool cards, gas pacs, home collection, etc. In one embodiment, oral samples, such as oral rinses, oral swabs etc. are collected e.g. to correlate the oral microbiome with the gut microbiome, or for other purposes.

After a sample is obtained, the types and/or the quantity (e.g. occurrence) in the sample of at least one microbe of interest is determined. In addition, a total amount of microbes may be determined, and then for each constituent microbe, a fractional percentage (e.g. relative amount, ratio, distribution, frequency, percentage, etc.) of the total is calculated. The result is typically correlated with at least one suitable control result, e.g. control results of the same parameter(s) obtained from healthy individuals (negative control), and/or individuals known to have a disease or condition of interest (positive control), or from subjects who have had the disease and condition of interest and are being or have been treated, either successfully or unsuccessfully, etc.

If a strong correlation between a condition of interest and only one or a few microbes has previously been established, it is possible that detection of their presence (or absence) alone will suffice to justify or suggest a conclusion that the individual being tested does or does not have a high risk of developing the condition of interest. In this case, detection may be done in any of a number of ways that are known to those of ordinary skill in the art, including but not limited to culturing the organism or the few organisms, conducting various analyses which are indicative of the presence of the microbe(s) of interest (e.g. by microscopy, using staining techniques, enzyme assays, antibody assays, etc.), or by sequencing of genetic material (DNA or RNA), and others. However, generally it will be beneficial to obtain as much information as possible (or at least more information) regarding the microflora present in the sample. Older techniques (e.g. cultivation) are generally impractical for such an undertaking. Thus, newer nucleic acid sequencing technology (NextGen or Xgen technology) is usually used. While any category (or categories) of nucleic acid(s) may be detected (usually amplified using, e.g. PCR techniques), particularly useful amplification strategies include the use of primers (e.g. universal primers) which amplify ribosomal RNA genes (rRNA). Such techniques and primers are well-known to those of skill in the art, e.g. see: Turnbaugh P J, Hamady M, Yatsunenko T, Cantarel B L, Duncan A, Ley R E, Sogin M L, Jones W J, Roe B A, Affourtit J P, Egholm M, Henrissat B, Heath A C, Knight R, Gordon J I (2009) A core gut microbiome in obese and lean twins. Nature 457(7228):480-484; Mai V, Draganov P-V (2009) Gillevet; and Gillevet, P. M. 2006. Multitag Sequencing and Ecogenomic Analysis, European patent application 07871488.8; International patent application PCT/US2007/084840; Recent advances and remaining gaps in our knowledge of associations between gut microbiota and human health. World journal of gastroenterology: WJG 15(1):81-5; Hattori M, Taylor T D (2009). The human intestinal microbiome: a new frontier of human biology. DNA research: an international journal for rapid publication of reports on genes and genomes 16(1):1-12; and Young V B, Schmidt T M (2008) Overview of the gastrointestinal microbiota. Advances in experimental medicine and biology 635:29-40. See also US patent applications 20090197249 and 20100143908, both to Gillevet, the complete contents of both of which are hereby incorporated by reference.

In some embodiments, what is determined is the distribution of microbial families within the microbiome. However, characterization may be carried to more detailed levels, e.g. to the level of genus and/or species, and/or to the level of strain or variation (e.g. variants) within a species, if desired (including the presence or absence of various genetic elements such as genes, the presence or absence of plasmids, etc.). Alternatively, higher taxanomic designations can be used such as Phyla, Class, or Order. The objective is to identify which microbes (usually bacteria, but also optionally fungi (e.g. yeasts), protists, etc.) are present in the sample from the individual and the relative distributions of those microbes, e.g. expressed as a percentage of the total number of microbes that are present, thereby establishing a microfloral pattern or signature for the individual being tested, e.g. for the region of the gut that has been sampled, or for the type of sample that is analyzed.

Once an individual patient's “signature” with respect to the targeted microbes has been determined, it is compared to known signatures obtained previously from control experiments. Such control experiments typically obtain “negative control” data from normal (healthy) individuals, i.e. comparable individuals who do not have disease symptoms, and positive control data from comparable individuals who do have the disease in question or did have at the time of the analysis. Based on a comparative analysis between the patient's signature and one or more reference or control signatures (and usually corroborated statistically by methods that are well-known to those of ordinary skill in the art) the likelihood or risk of the patient for developing the disease of interest is determined and thus can be used as a predictive diagnostic. For example, a person with a signature that is not similar to or within the range of values seen in normal control signatures, but which is more similar to or within ranges determined for positive controls, may be deemed to be at high risk for developing the disease. This is generally the case, for example, if his/her level or amount of at least one correlatable microbe is associated with the disease state with a statistically significant (P value) of less than about 0.05. Alternatively, for patients who are already symptomatic, a previous diagnosis may be corroborated, and/or an explanation of symptoms may be provided.

In other embodiments of the invention, when many taxa are being considered, the overall pattern of microflora is assessed, i.e. not only are particular taxa identified, but the percentage of each constituent taxon is taken in account, in comparison to all taxa that are detected and, usually, or optionally, to each other. Those of skill in the art will recognize that many possible ways of expressing or compiling such data exist, all of which are encompassed by the present invention. For example, a “pie chart” format may be used to depict a microfloral signature; or the relationships may be expressed numerically or graphically as ratios or percentages of all taxa detected, etc. Further, the data may be manipulated so that only selected subsets of the taxa are considered (e.g. key indicators with strong positive correlations). Data may be expressed, e.g. as a percentage of the total number of microbes detected, or as a weight percentage, etc.

In one embodiment, a nonparametric multivariate test such as Metastats, Analysis of Similarity, Principle Component Analysis, Non-Parametric MANOVA (Kruskal-Wallace) etc.

can be used to associate microbiome dysbiosis with a statistical significant (P value) of less than 0.05. Such tests are known in the art and are described, for example, by White J R, Nagarajan N, Pop M (2009) Statistical Methods for Detecting Differentially Abundant Features in Clinical Metagenomic Samples. PLoS Computational Biology 5(4):1-11; and Clarke K R, Gorley R N (2001) PRIMER v5: User Manual and Tutorial, PRIMER-E Ltd. Plymouth Marine Laboratory, UK.

In other embodiments, phylogenetic methods such as Unifrac can be used to associate microbiome dysbiosis with the disease state with a statistically significant (P value) of less than 0.05. See, for example, Lozupone C, Knight R (2005) UniFrac: a new phylogenetic method for comparing microbial communities. Appl Environ Microbiol 71:8228-8235,

In other embodiments, support vector machines can be used to associate microbiome dysbiosis with a disease state with sufficiently high classification measure (F-measure) and appropriate sensitivity and specificity that is accepted in the state of the art. See, for example,Yang C, Mills D, Mathee K, Wang Y, Jayachandran K, Sikaroodi M, Gillevet P, Entry J, Narasimhan G (2006) An ecoinformatics tool for microbial community studies: Supervised classification of Amplicon Length Heterogeneity (ALH) profiles of 16S rRNA. Journal of Microbiological Methods 65(l):49-62.

In other embodiments, correlation network and correlation difference network methods can be used to associate microbiome dysbiosis with the disease state with a statistical significant (P value) of less than 0 05. See, for example, Weckwerth W, Loureiro M E, Wenzel K, Fiehn O (2004) Differential metabolic networks unravel the effects of silent plant phenotypes. PNAS 101(20):7809-7814.

Once a patient is identified as having, or as at high risk for developing, a disease or condition, suitable clinical intervention can be undertaken to alter the identity and/or the relative abundance of gut microflora in the individual. Accordingly, the present invention also encompasses the identification of suitable therapeutic targets for intervention and the selection/development of suitable treatment protocols. Exemplary treatments include but are not limited to: eliminating or lessening microflora associated with the condition e.g. using antibiotics or other therapies, for example, therapies that are specific for eliminating or lessening the number of targeted microflora, without affecting or minimally affecting desirable microflora, if possible; or increasing microflora that compete with the unwanted microflora, and/or which are correlated with a lack of disease symptoms, e.g. by administering probiotic and/or prebiotic supplements; by microflora) transplants (e.g. from healthy donors); by dietary modifications; by lifestyle modifications (such as increasing exercise, eliminating unhealthy behaviors such as excessive alcohol consumption, eliminating smoking, regulating sleep habits, decreasing or coping with stress, eliminating recreational drug use, etc.); by changes of diet to eliminate or lessen intake of highly processed foods; by administering probiotic substances (e.g. yogurts, kefir, fermented milk, etc.); by increasing intake of prebiotic nutrients (e.g. fructooligosaccharides such as oligofructose and inulin; galactooligosaccharides (GOS), lactulose, mannan oligosaccharides (MOS), etc., either from natural sources or in prepared forms); etc.

In one embodiment of the invention, the disease that is of interest is HE, particularly HE that develops in patients with liver disease such as cirrhosis. The cirrhosis may have any of a number of different causes, or more than one cause, including but not limited to alcoholism (Alcoholic liver disease or ALD), non-alcoholic steatohepatitis (NASH), chronic hepatitis C, chronic hepatitis B, primary biliary cirrhosis, primary sclerosing cholangitis, autoimmune hepatitis, hereditary hemochromatosis, Wilson's disease, alpha 1-antitrypsin deficiency, cardiac cirrhosis, galactosemia, glycogen storage disease type IV, cystic fibrosis, hepatotoxic drugs or toxins, lysosomal acid lipase deficiency (LAL Deficiency), idiopathic (i.e. unknown) causes, etc.

The studies described herein have demonstrated that the presence and/or absence and/or relative abundance of certain genera of bacteria are associated with liver diseases such as cirrhosis, and with conditions or complications associated with liver disease, e.g. cognitive impairment (encephalopathy), inflammation, endotoxemia, endothelial dysfunction, etc. In exemplary embodiments, in stool samples, bacteria such as Porphyromonadaceae and Alcaligeneceae are associated with cognitive impairment; bacteria such as Enterobacteriaceae, Veillonellaceae and Fusobacteriaceae are associated with inflammation; and bacteria such as Ruminococcaceae have a negative correlation endotoxemia; and in mucosal samples, bacteria such as Enterococcus, Burkholderia and Veillonellaceae are associated with HE; Alcaligeneceae and Porphyromonadaceae are associated with poor cognition; and Roseburia, Lachnosperaceae, Ruminococeaceae and Inertae Sedis XIV are associated with better cognition.

Further embodiments of the invention provide methods for determining reference microfloral signatures, and databases comprising the same. Such signatures constitute prototypes or models for use as references when the assessment of an individual's microflora is undertaken. In some embodiments, control signatures are collected and averaged or amalgamated to develop reference signatures which are correlated with a disease or condition of interest (and/or with the absence of the disease/condition). The reference signatures may be in the form of e.g. sequences which are characteristic of particular bacterial types, according to any useful classification (phylum, order, class, genus, species, strain, type, etc.) Further, the reference signatures generally include this information for relevant groups and subgroups of microflora, e.g. those associated with a particular disease, condition, etc. The characteristics of the reference signatures are generally recorded (stored, compiled, etc.) in an electronic computerized catalog, library, database, etc. that is accessible to a practitioner of the invention. Such databases may include the Ribosomal Database versions 8 and 10, Greengenes, and Genbank. The invention also encompasses computer programs (e.g. executable software programs, and/or computers configured to carry out the programs), which enable a practitioner to enter analytical data into the system (e.g. the results of rRNA PCR amplification of a stool sample, which may be the patient's “signature”) and to carry out a comparison to the stored reference signatures. Output from the program may include an expression of the level of similarity between the patient's signature and one or more relevant stored reference signatures, and/or the statistical likelihood that the patient already has or is likely to develop a disease or condition associated with one or more reference signatures.

In some embodiments, the gut “signature” of a subject includes (or is) the results of an analysis of the metabolome of the subject. In other words, instead of, or in addition to, determining the identity, the presence or absence, and/or relative abundance of bacteria (or other microflora) in the gut, the identity, presence or absence and/or relative abundance of selected micofloral metabolic products of interest is determined. Exemplary metabolites include but are not limited to indoles, oxindoles, short chain fatty acids, amino acids, bile acids and inflammation. The metabolites may be associated with (e.g. characteristic of) one or more bacterial (or other microfloral) taxa of interest. Exemplary metabolites that may be included in such a gut metabolome signature include but are not limited to volatile organic compounds detected by GC-MS, and hydrophobic and hydrophilic organic compounds detected by LC-MS. In some embodiments, nuclear magnetic resonance (NMR) is utilized for detection. Other detectable metabolites are known to those of skill in the art.

In yet other embodiments of the invention, the gut signature of a subject may be, or may include, results obtained by analyzing the protein content of a biological sample (e.g. a gut sample), of a subject. The results may include the identity of the proteins, the presence or absence of selected proteins, the relative abundance of the proteins (e.g. compared to suitable controls), etc. The proteins may be associated with (e.g. characteristic of) one or more bacterial (or other microfloral) taxa of interest. Exemplary proteins that may be included in such a gut proteome signature include but are not limited to those which are known to those of skill in the art.

The invention also provides methods for developing suitable treatment protocols for patients with liver disease. The methods involve determining a gut microbiome signature using a biological sample from the patient as described herein, and interpreting the signature by correlating the results with the presence and/or likelihood of developing a condition of interest associated with liver disease. Conditions of interest that can be detected, confirmed, or prognosticated using this method include but are not limited to encephalopathy, inflammation, endotoxemia, endothelial dysfunction, status of liver function, and/or the likelihood of the worsening or severity of symptoms, e.g. development of hepatic encephalopathy, cognitive dysfunction, coma, renal failure, death and need for liver transplant etc. Once such an analysis has been completed, it will encourage and delineate paths for suitable therapy developed or designed to combat, treat, lessen symptoms of, etc. the conditions that are identified. The microbial signature will change and be able to predict response to general therapy for hepatic encephalopathy (probiotics, pre-biotics, rifaximin, lactulose, lactitol, metronidazole, neomycin, zinc and other antibiotics) and for specific therapy for the chronic liver disease ;therapy protocol may include but are not limited to: anti-viral therapy for hepatitis B, C and/or D; weight loss therapy and/or surgery for non-alcoholic liver disease and obesity-associated liver disease; alcohol abstinence for alcoholic liver disease; therapy for Wilson's disease; alpha-1 anti-trypsin repletion; specific S therapies for hepatic encephalopathy (e.g. pre- and probiotic administration, antibiotic administration, etc.); liver transplantation; etc.

The invention also provides methods for monitoring the efficacy of a treatment protocol that is ostensibly treating a condition or complication associated with liver disease. The method involves determining gut microbiome signatures of a patient who is or who is going to be treated for liver disease or for a condition or complication of interest associated with liver disease, e.g. encephalopathy, inflammation, endotoxemia, endothelial dysfunction. Multiple signatures are generally obtained and analyzed at suitable time intervals, e.g. just prior to treatment to establish a baseline, and then repeatedly every few days, weeks or months thereafter. Subsequent signatures are compared to suitable reference signatures and/or to one or more previous signatures from the patient. If subsequent signatures indicate that the patient's gut microfloral signature is improving (e.g. is more similar to that of controls who do not have the condition of interest, especially when compared to previous patient signatures) then the treatment may be continued without adjustment, or may be gradually decreased, and may even be discontinued. However, if no improvement is observed, or if a signature indicates a worsening of the condition, then the treatment protocol can be adjusted accordingly, e.g. more of a treatment agent may be administered, or a different and/or more drastic form of treatment may be implemented, etc. The microflora signature is thus used to assess treatment adequacy, treatment response and the recovery of e.g. brain function after therapies such as those available for liver disease.

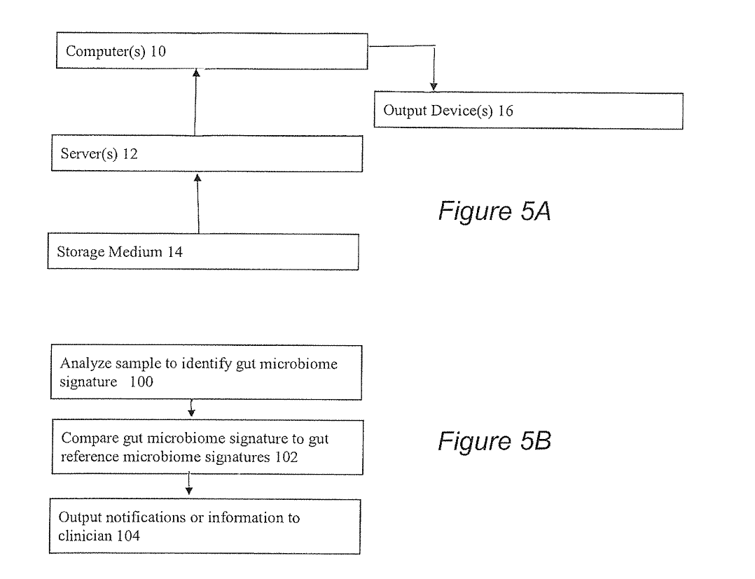

FIGS. 5A-B show in simplified form that the invention can be best practiced using one or more computers/data processors 10 which receive and/or produce data defining a gut microbiome signature for a patient based on one or more samples obtained from the patient (analysis step 100 provides for the determination of the microbiome signature for the patient). The microbiome signature for the patient is compared, preferably using at least one of the one or more computers/data processors 10 with gut microbiome reference signatures (analysis step 102 provides for a computer based comparison). The microbiome reference signatures include either or both one or more positive gut microbiome reference signature(s) based on results from control subject(s) with encephalopathy, and one or more negative gut microbiome reference signature(s) based on results from control subject(s) without encephalopathy. The gut microbiome reference signatures may be stored on one or more servers 12 or the one or more computers 10, and will generally be stored in a non-transient medium 14 such as a hard disk, programmable read only memory (PROM), compact disc (CD), DVD, or other storage device, and be used multiple times for comparison purposes with multiple patients or for comparisons with samples taken from the same patient over a period of time to monitor the efficaciousness of the treatment protocol. A clinician is preferably provided with output from the one or more computers 12 on an output device 16 such as a computer display, printer, display of a mobile telephone, iPad, or other tablet, or other suitable device which will notify the clinician or provide the clinician with information from which he or she can make relevant decisions on risk of developing a disease, identification of one or more suitable treatment protocols, being able to deduce the effectiveness/non-effectiveness of a therapy, etc. (output step 104 shows presentation of the information from the comparison, notification of risks, appropriate alarms, etc.). For example, the computer(s) will be programmed to provide for one or more statistical analysis methods. If the gut microbiome signature for the patient statistically significantly matches a positive gut microbiome reference signature, the clinician might be notified as output an indication or information from which the clinician can deduce that the patient is at risk of developing encephalopathy. If the gut microbiome signature for the patient statistically significantly matches a negative gut microbiome reference signature, then the clinician might be notified as output an indication or information from which the clinician can deduce that the patient either does not have or is not at risk of developing encephalopathy. As described above, the system and method may provide as output identification of one or more treatment protocols for a patient, or may be used to monitor the effectiveness of a treatment/therapy over time. In operation, the computers 10, servers 112, storage medium 114, and output devices 116 may be used together or may be remote from one another and can communication through a network such as the Internet.

The following Examples describe various embodiments of the invention, but should not be interpreted as limiting the invention in any way.

### EXAMPLES

### Example 1

### Linkage of Gut Microbiome with Cognition in Hepatic Encephalopathy

### Abstract

Background/aims: Hepatic encephalopathy (HE) has been related to gut bacteria and inflammation in the setting of intestinal barrier dysfunction. We proposed to link the gut microbiome with cognition and inflammation in HE using a systems biology approach. Methods: Multi-tag pyrosequencing (MTPS) was performed on stool of cirrhotics and age-matched controls. Cirrhotics with/without HE underwent cognitive testing, inflammatory cytokines, and endotoxin analysis. HE patients were compared to those without HE using a correlation network analysis. A select group of HE patients (n=7) on lactulose underwent stool MTPS before and after lactulose withdrawal over 14 days. Results: 25 patients [17 HE (all on lactulose, 6 also on rifaximin) and 8 no HE, age 56±6 years, MELD 16±6] and 10 controls were included. Fecal microbiota in cirrhotics was significant different (higher Enterobacteriaceae, Alcaligeneceae, Fusobacteriaceae and lower Ruminococcaceae and Lachnospiraceae) compared to controls. We found altered flora (higher Veillonellaceae), poor cognition, endotoxemia and inflammation (IL-6, TNF-α, IL-2 and IL-13) in HE compared to cirrhotics without HE. In the cirrhosis group, Alcaligeneceae and Porphyronionadaceae were positively correlated with cognitive impairment. Fusobacteriaceae, Veillonellaceae and Enterobacteriaceae were positively and Ruminococcaceae negatively related to inflammation. Network analysis comparison showed robust correlations (all p<1E-5) only in the HE group between the microbiome, cognition and IL-23, IL-2 and IL-13. Lactulose withdrawal did not change the microbiome significantly beyond Fecalibacterium reduction. Conclusions: Cirrhosis, especially HE, is associated with significant alterations in the stool microbiome compared to healthy individuals. Specific bacterial families (Alcaligeneceae, Porphyromonadaceae, Enterobacteriaceae) are strongly correlated with cognition and inflammation in HE.

### Introduction:

Cirrhosis is often complicated by hepatic encephalopathy (HE), a condition characterized by cognitive impairment and poor survival (2, 8). There is evidence that pathogenic abnormalities in HE are related to the gut flora and their by-products such as ammonia and endotoxin in the setting of intestinal barrier dysfunction and systemic inflammation (14, 35, 36, 44). Patients with cirrhosis also have widespread derangements of their immune response, which can potentiate insults such as sepsis and result in HE (36, 43). The current treatments for HE rely on manipulation of the gut flora, however, their mechanisms of action as well as prediction of resistance to therapy are not clear (2). In addition, the characterization of gut flora in prior HE studies has been limited by the use of culture-based techniques that do not identify the majority of the intestinal bacteria (23). Since the pathogenesis of HE likely spans several metabolic processes, we proposed that a systems biology approach could be useful to identify novel functional hypotheses and new therapeutic targets for HE. Specifically, we used correlation network analysis to correlate features within each treatment group in order to dissect out functionality in the system (27). This analysis provides potential clues to the functionality of the system leading the way to hypothesis-driven experimental research.

The aims of this study were (a) to link the gut microbiome with cognition and inflammation in cirrhotic patients with and without HE using a systems biology approach (b) identify differences in the microbiome of healthy controls and cirrhotic patients and (c) define the effect of lactulose withdrawal on microbiome of cirrhotic patients. The a priori hypothesis was that the gut microbiome composition would be correlated with cognition and inflammation in cirrhotic patients with HE and that this association would be different from those who have never developed HE.

METHODS: Patients with cirrhosis and healthy age-matched controls were recruited after a written informed consent. We only included controls without liver disease and those who were not taking medications apart from those for hypertension, hyperlipidemia or gastro-esophageal reflux disease. In the case of cirrhotic patients, we excluded those with a current infection (defined by elevated WBC count, clinical suspicion or fever), who had experienced variceal bleeding within the last 4 weeks, on gut-absorbable antibiotic therapy, or had alcohol or illicit drug intake within 3 months (checked by drug and alcohol screens). The data collected from their medical record were MELD score, etiology of cirrhosis, complications of cirrhosis in the past, and current medication use. Patients in the “no HE” group had never had an episode of HE and were not on any therapy for it. Patients in the “HE” group had suffered at least one HE episode within the last 3 months and were currently controlled on lactulose alone or lactulose with rifaximin. We did not include patients during an acute HE episode because those patients are often hospitalized, are on systemic antibiotic therapy, and are not able to give consent or perform cognitive testing.

All subjects underwent a mini-mental status exam and only those scoring above 25 were included in the full study (11). Participants then underwent a 24-hour dietary recall. Subsequently a recommended cognitive battery consisting of the following tests was administered to the cirrhotic patients; (a) Psychometric hepatic encephalopathy score (PHES), (b) block design test (BDT: subjects are required to replicate standardized designs with given blocks in a timed manner. The score is based on the designs correctly copied) and (c) Inhibitory control test. [This is a 15 minute computerized test. Subjects are instructed to respond to alternating presentations of X and Y on the screen (targets) while inhibiting response when X and Y are not alternating (lures)] (3, 41). The PHES consists of 5 tests: number connection test-A/B (NCT-A/B: subjects are asked to “join the dots” between numbers or numbers and alphabets in a timed fashion and the number of seconds required is the outcome), digit symbol (DST: subjects are required to copy corresponding figures from a given list within 2 minutes and the number correctly copied is the result), line drawing [LDTt (time) and LDTe (errors): subjects are required to trace a line between two parallel lines and balance between speed and accuracy. Time required and the number of times the subject's line strays beyond the marked lines (errors) are recorded] and serial dotting (SDT, subjects are asked to dot the center of a group of blank circles and the time required is the outcome)]. The PHES is a validated battery for cognitive dysfunction in cirrhosis and tests for psychomotor speed, visuo-motor coordination, attention and set-shifting(32). The BDT tests for visuo-motor coordination. The ICT is a validated computerized test of attention, psychomotor speed, response inhibition and working memory. A high score on BDT, DST and ICT targets and a low score on the rest of the tests indicates good cognitive performance. Cirrhotic patients also underwent serum collection for inflammatory cytokines testing for innate immunity [IL-1b, IL-6, TNF-α (tumor necrosis factor-alpha)], Th1 response ([IFN-γ (interferon-gamma) and IL-2], Th2 response (IL-4, IL-10, IL-13], Th17 response (IL-17 and IL-23), and endotoxin. These were analyzed in duplicate by multiplex bead-based sandwich ELISA and LAL assay for endotoxin using published techniques by AssayGate Inc, Ijamsville, Md. (4, 17, 45).

Prospective study: A selected group of seven cirrhotic patients in the HE group currently only on lactulose (who were also included in the cross-sectional study) were systematically withdrawn from therapy. Their diet was controlled over the study period. Their stool microflora was analyzed while on lactulose and then 14 days off of lactulose therapy. Day 14 was chosen since prior culture-based studies have shown a change in fecal flora after lactulose initiation within that time frame (31).

Interrogation of the Microbiome: Stool was collected and DNA extracted for microbiome analysis within 24 hours of collection from patients and controls using published techniques (29). We first routinely use Length Heterogeneity PCR (LH-PCR) fingerprinting of the 16S rRNA to rapidly survey our samples and standardize the community amplification. We then interrogated the microbial taxa associated with the gut fecal microbiome using Multitag Pyrosequencing (MTPS). This technique allows the rapid sequencing of multiple samples at one time yielding thousands of sequence reads per sample (12).

Microbiome Community Fingerprinting: LH-PCR was done to standardize the community analysis as previously published (21). Briefly, total genomic DNA was extracted from tissue using Bio101 kit from MP Biomedicals Inc., Montreal, Quebec as per the manufacturer's instructions. About 10 ng of extracted DNA was amplified by PCR using a fluorescently labeled forward primer 27F (5’-(6FAM) AGAGTTTGATCCTGGCTCA G-3′, SEQ ID NO: 1) and unlabeled reverse primer 355R′ (5′-GCTGCCTCCCGTAGGAGT-3′, SEQ ID NO: 2). Both primers are universal primers for Bacteria (22). The LH-PCR products were diluted according to their intensity on agarose gel electrophoresis and mixed with ILS-600 size standards (Promega) and HiDi Formamide (Applied Biosystems, Foster City, Calif.). The diluted samples were then separated on a ABI 3130x1 fluorescent capillary sequencer (Applied Biosystems, Foster City, Calif.) and processed using the Genemapper™ software package (Applied Biosystems, Foster City, Calif.). Normalized peak areas were calculated using a custom PERL script and OTUs constituting less than 1% of the total community from each sample were eliminated from the analysis to remove the variable low abundance components within the communities.

MTPS: We employed the MTPS process to characterize the microbiome from the fecal samples. Specifically, we have generated a set of 96 emulsion PCR fusion primers that contain the 454 emulsion PCR linkers on the 27F and 355R primers and a different 8 base “barcode” between the A adapter and 27F primer. Thus, each fecal sample was amplified with unique bar-coded forward 16S rRNA primers and then up to 96 samples were pooled and subjected to emulsion PCR and pyrosequenced using a GS-FLX pyrosequencer (Roche). Data from each pooled sample were “deconvoluted” by sorting the sequences into bins based on the barcodes using custom PERL scripts. Thus, we were able to normalize each sample by the total number of reads from each barcode. We have noted that ligating tagged primers to PCR amplicons distorts the abundances of the communities and thus it is critical to incorporate the tags during the original amplification step (12). Several groups have employed various barcoding strategies to analyze multiple samples and this strategy is now well accepted (38).

RDP10 Analysis: We identified the taxa present in each sample using the Bayesian analysis tool in Version 10 of the Ribosomal Database Project (RDP10). The abundances of the bacterial identifications were then normalized using a custom PERL script and taxa present at >1% of the community were tabulated. We chose this cutoff because of our a priori assumption that taxa present in <1% of the community vary between individuals and have minimal contribution to the functionality of that community and 2,000 reads per sample will only reliably identify community components that are greater than 1% in abundance (13).

This study was approved by the Institutional Review Boards of the McGuire VA Medical Center and the Virginia Commonwealth University Medical Center in Richmond. Statistical analysis: Clinical and microbiome features of controls were compared to patients with cirrhosis with Metastats using the p-value and the false discovery rate (q-value) for non-normal distributions (42). A principal coordinate analysis was also used to show differences between the two groups. Only taxa with average abundances greater than one percent, p-values <0.05 and low q-values (i.e. low risk of false discovery) was considered significant.

The cirrhosis group was divided into those with and without HE and were compared. Data from the significant variables between HE and non-HE groups were combined in a MANOVA model. Within HE patients, comparison was made between those on lactulose alone to those with lactulose and rifaximin. Microbiome abundance comparisons between groups were made at a family level using non-parametric tests. A comparison was performed between patients on and withdrawn off of lactulose therapy using the Wilcoxon matched-pair signed rank tests. All values are presented as mean±standard deviation unless mentioned otherwise.

Correlation Network Models: Groups were divided into HE or no HE and they were analyzed separately. The microbiome features along with the presence of HE, cirrhosis severity, serum markers and cognitive function tests were correlated using a Pearson's correlation function and then filtered for correlations greater than 0.90. These correlations were calculated using a custom R module and the correlations and corresponding attributes were imported into Cytoscape for visualization of the network models (34). We then compared the network topology of the two disease classes, HE and no HE, to identify which sub-networks were present in one and not the other giving us clues on system functionality. It is assumed that correlations present in one treatment group that are missing in another not only differentiate the groups but indicate potential clues to the functionality of the system leading the way to hypothesis-driven experimental research.

Results: Twenty five cirrhotic patients (MELD score 16±6) and ten healthy controls were included (Table 1). All patients and controls were non-vegetarians and had similar dietary intake and constituents on recall prior to sample collection (mean intake 2470 Kcal and 16% protein intake). Patients had been abstinent of alcohol and illicit drugs for at least 3 months confirmed by serum alcohol and urine drug screens. At the time of sample collection, none of the subjects had systemic infections as evidenced by normal WBC counts, normal body temperature and physical examination unremarkable for infections. The majority of patients and none of the controls were on proton pump inhibitor (PPI) therapy (92%) (Table 2). Thirteen (52%) had alcoholic liver disease, rest had hepatitis C (40%) or cryptogenic cirrhosis (8%); 8 had both alcoholic and HCV disease. HE was present in 17 patients (68%; 11 were on lactulose alone, 6 were on both lactulose and rifaximin). None of the HE patients were on rifaximin alone. All patients who were on rifaximin were started on it due to difficulties in tolerating lactulose alone. All patients in the HE group had residual cognitive impairment or minimal HE at the time of the testing (5, 30).

TABLE 1
Baseline characteristics of the groups

| | Controls | Cirrhosis with HE | Cirrhosis without HE |
|---|---|---|---|
| | (n = 10) | (n = 17) | (n = 8) |
| Age (years) | 54 ± 5 | 56 ± 3 | 55 ± 5 |
| Gender (Men/Women) | 6/4 | 16/1 | 7/1 |
| Ethnicity (Caucasian/ | 6/3/1 | 11/5/1 | 5/2/1 |
| African-American/ | | | |
| Hispanic) | | | |
| BMI | 25 ± 3 | 26 ± 5 | 25 ± 3 |

TABLE 2
Features of patients with and without hepatic encephalopathy

| | HE (n = 17) | No HE (n = 8) | P value |
|---|---|---|---|
| Alcoholic etiology | 58% | 37% | 0.41 |
| Prior variceal bleeding | 23% | 0% | 0.07 |
| Prior SBP | 0% | 0% | 1.0 |
| Renal insufficiency | 0% | 18% | 0.09 |
| Clinically evident ascites | 29% | 47% | 0.65 |
| Median daily bowel | 2 | 1 | 0.02 |
| movements | | | |
| Proton pump inhibitor therapy | 94% | 86% | 0.51 |
| MELD score | 17 ± 6 | 12 ± 5 | 0.048 |
| Venous ammonia | 52 ± 28 | 31 ± 21 | 0.148 |
| WBC count (/mm³) | 5.2 ± 2 | 5 ± 3 | 0.33 |
| Endotoxin | 0.27 ± 0.24 | 0.059 ± 0.012 | 0.002 |
| IL-1b (pg/ml) | 6.2 ± 11.1 | 1.07 ± 0.55 | 0.06 |
| IFN-γ (pg/ml) | 11.3 ± 26.6 | 1.6 ± 1.8 | 0.148 |
| IL-10 (pg/ml) | 8.21 ± 8.70 | 2.9 ± 1.5 | 0.022 |
| IL-23 (pg/ml) | 1842 ± 4873 | 317 ± 359 | 0.205 |
| IL-17 (pg/ml) | 32.1 ± 81.3 | 4.53 ± 5.27 | 0.107 |
| IL-6 (pg/ml) | 67.8 ± 72.2 | 9.3 ± 7.8 | 0.004 |
| IL-2 (pg/ml) | 48 ± 91 | 2.7 ± 2 | 0.04 |
| TNF-α (pg/ml) | 7.01 ± 4.09 | 4.33 ± 2.33 | 0.05 |
| IL-13 (pg/ml) | 32.0 ± 17.2 | 0.80 ± 0.02 | 0.0001 |

There were significant differences at baseline between those with and without HE with respect to endotoxemia and inflammation; all of which were significantly worse in HE patients. As expected, HE patients had a significantly higher MELD score and a higher number of daily bowel movements since they were on lactulose. MELD: model for end-stage liver disease, HE: hepatic encephalopathy.

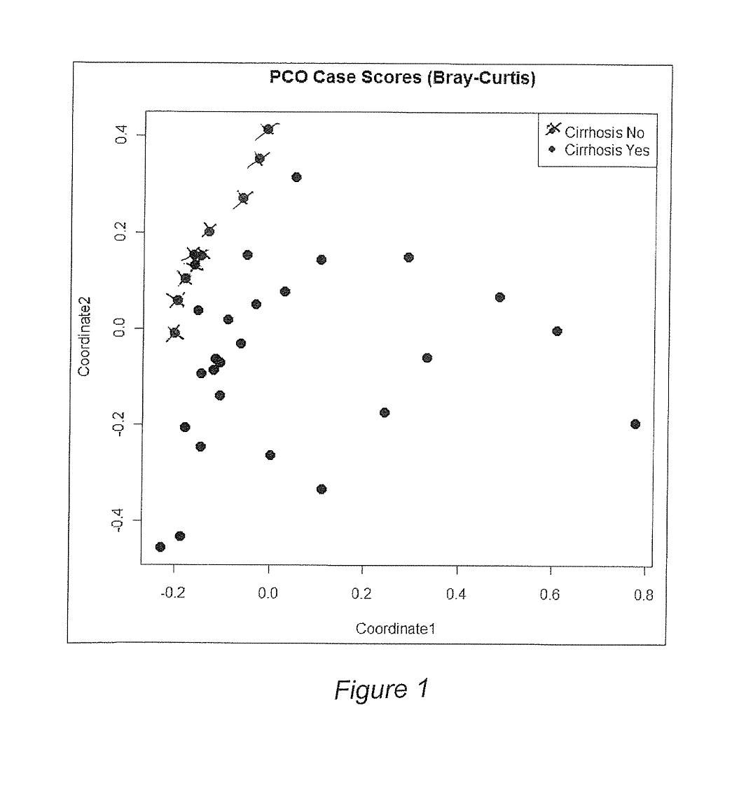

Cross-sectional microbial analysis between controls and patients with cirrhosis: There were significant differences in stool microbiome between cirrhotic patients and controls (FIG. 1, Table 3). The abundances of the taxa in the controls were Actinobacter (Coriobacteriaceae 1%), Firmicutes (Lachnospiraceae 23%, Ruminococcaceae 17%, Veillonellaceae 3%, Streptococcaceae <1%, Leuconostocaceae <1%, Lactobacillaceae <1%, Clostridiaceae <1%, Enterococcaceae <1% and Erysipelothrixaceae <1%), Bacterioidetes (Bacterioideceae 27%, Prevotellaceae 8%, Porphyromonadaceae 6%, Rickenellaceae <1%), Fusobacteria (<1%), Proteobacteria (Enterobacteriaceae <1%, Alcaligenaceae <1%, Pasteurellaceae <1%, Burkholderiaceae <1% and Moraxellaceae <1%) and 6% of uncertain placement. The diversity of the microbial phyla in the cirrhotic group was: Actinobacter: (Coriobacteriaceae 16%), Firmicutes (Lachnospiraceae 80%, Rutninococcaceae 68%, Veillonellaceae 60%, Streptococcaceae 40%, Leuconostocaceae 36%, Lactobacillaceae 8%, Clostridiaceae 8%, Enterococcaceae 4% and Elysipelothrixaceae 2%), Bacterioidetes (Bacterioideceae 88%, Prevotellaceae 44%, Porphyrotnonadaceae 44%, Rickenellaceae 36%), Fusobacteria (16%), Proteobacteria (Enterobacteriaceae 40%, Alcaligenaceae 49%, Pasteurellaceae 12%, Burkholderiaceae 4% and Moraxellaceae 4%) and 44% of uncertain placement. There was a significantly higher abundance of Lachnospiraceae and Ruminococceae in the control group while Enterobacteriaceae, Fusobacteriaceae, Alcaligenaceae, Lactobacillaceae and Leuconostocaceae were significantly lower in the controls compared to cirrhotic patients. These differences persisted and widened when controls were compared to patients with and without HE (Table 4a and 4b). The differences existed for Leuconostocaceae, Clostridialesincertae Sedis XIV, Fusobacteriaceae, Lachnospiraceae, Ruminococcaceae in both groups of cirrhotic patients (with or without HE). Interestingly however, the HE group differed from controls on several additional bacterial families compared to cirrhotics without HE in that they had a significantly higher concentration of Enterobacteriaceae, Alcaligenaceae, Lactobacilaceae and Streptococcaceae.

TABLE 3
Differences in bacterial abundances between
controls and cirrhotic patients

| | Control | | Cirrhosis | | | |
|---|---|---|---|---|---|---|
| | Mean | SEM | Mean | SEM | P value | Q value |
| Leuconostocaceae | 0.00 | 0.00 | 2.02 | 0.70 | 0.0009 | 0.009 |
| Clostridium | 7.35 | 1.59 | 1.08 | 0.18 | 0.0009 | 0.008 |
| Incertae | | | | | | |
| sedis XIV | | | | | | |
| Lachnospiraceae | 23.44 | 2.24 | 10.40 | 2.60 | 0.0009 | 0.008 |
| Ruminococcaceae | 17.72 | 1.89 | 6.75 | 1.28 | 0.001 | 0.008 |
| Enterobacteriaceae | 0.00 | 0.00 | 7.60 | 2.89 | 0.001 | 0.008 |
| Fusobacteriaceae | 0.00 | 0.00 | 1.80 | 1.06 | 0.0059 | 0.0408 |
| Alcaligenaceae | 0.89 | 0.34 | 2.76 | 0.73 | 0.032 | 0.1723 |

A comparison between controls and cirrhotic patients' microbial flora was performed using Metastats and only significantly different and with values around 1% are shown; the rest were non-significant. Q value indicates the false discovery rate and a lower value is generally preferred to avoid a false positive result. There was a significantly higher abundance of Lachnospiraceae and Runfinococceae in the control group while Enterobacteriaceae, Fnsobacteriaceae, Alcaligenaceae, Lactobacillaceae and Leuconostocaceae were significantly lower in the controls compared to patients with cirrhosis. Incertae sedis: uncertain placement; SEM: standard error of mean

TABLE 4a
Differences in bacterial abundances between controls
and cirrhotic patients with HE

| | Control | Control | HE | HE | | |
|---|---|---|---|---|---|---|
| Name | mean | SEM | mean | SEM | pvalue | Qvalue |
| Leuconostocaceae | 0.00 | 0.00 | 2.19 | 1.08 | 0.0009 | 0.007 |
| Clostridiales_Incertae | 7.35 | 1.59 | 0.99 | 0.21 | 0.0009 | 0.007 |
| Sedis XIV | | | | | | |
| Ruminococcaceae | 17.72 | 1.89 | 5.68 | 1.42 | 0.0009 | 0.007 |
| Lachnospiraceae | 23.44 | 2.24 | 9.54 | 3.72 | 0.0039 | 0.022 |
| Enterobacteriaceae | 0.00 | 0.00 | 10.02 | 4.13 | 0.0049 | 0.026 |
| Streptococcaceae | 0.62 | 0.26 | 4.05 | 1.98 | 0.0239 | 0.099 |
| Fusobacteriaceae | 0.00 | 0.00 | 1.36 | 0.95 | 0.0369 | 0.146 |
| Alcaligenaceae | 0.89 | 0.34 | 2.61 | 0.74 | 0.048 | 0.169 |

Table 4a shows the differences between bacterial abundances in stool of controls and patients with HE; only those bacteria whose abundances were >1% and were significantly different between groups are shown. There was a significantly higher abundance of Enterobacteriaceae, Fusobacteriaceae, Leuconostocaceae, Streptococcaceae and Alcaligenaceae in HE patients while the rest of the bacteria listed were lower in the HE group. Incertae sedis: uncertain placement; SEM: standard error of mean SEM: standard error of mean

TABLE 4b
Differences in bacterial abundances between controls
and cirrhotic patients without HE

| | | | No | No | | |
|---|---|---|---|---|---|---|
| | Control | Control | HE | HE | P | Q |
| | Mean | SEM | mean | SEM | value | value |
| Leuconostocaceae | 0.00 | 0.00 | 1.69 | 0.95 | 0.0001 | 0.001 |
| Clostridiales_Incertae | 7.35 | 1.59 | 1.29 | 0.35 | 0.0001 | 0.001 |
| Sedis XIV | | | | | | |
| Fusobacteriaceae | 0.00 | 0.00 | 2.75 | 2.75 | 0.0001 | 0.001 |
| Lachnospiraceae | 23.44 | 2.245 | 12.08 | 2.47 | 0.003 | 0.002 |
| Ruminococcaceae | 17.72 | 1.89 | 9.04 | 7.62 | 0.019 | 0.008 |

Table 4b shows the differences between bacterial abundances in stool of controls and cirrhotic patients without HE; only those bacteria whose abundances were >1% and were significantly different between groups are shown. There was a significantly higher abundance of Fusobacteriaceae and Leuconostocaceae in cirrhotic patients without HE while the rest of the bacteria listed were lower in the cirrhotic no HE group. Incertae sedis: uncertain placement; SEM: standard error of mean SEM: standard error of mean

Cross-sectional analysis within the cirrhosis group: MELD was not correlated with endotoxin, inflammatory cytokines or cognition. We also did not find any differences in the inflammatory cytokines or endotoxemia between cirrhotic patients of differing etiologies using ANOVA, possibly due to the sample size, dual etiologies and probable effect of HE overwhelming the underlying etiologies (data not shown). Interestingly, MELD score was positively correlated with Enterobacteriaceae (r=0.61, p=0.001) and negatively with Ruminococcaceae (r=−0.38, p=0.05) with a trend towards lower Prevotellaceae (r=−0.36, p=0.056). Enterobacteriacae were also associated with TNF-α (r=0.5, p=0.03). Veillonellaceae and Fusobacteriaceae were also associated with worsening inflammation (IL-13, IL-6) and endotoxemia (p<0.05). Ruminococcaceae, importantly, were negatively correlated with endotoxemia p=0.02). The presence of Alcaligeneceae and Porphyromonadaceae was associated with poor cognition on individual tests (Table 5).

TABLE 5
Correlation between poor cognitive performance and presence of
Alcaligeneceae and Porphyromonadaceae in the entire group

| | Alcaligeneceae | | Porphyromonadaceae | |
|---|---|---|---|---|
| Cognitive tests | R | P value | R | P value |
| Higher value indicates poor | | | | |
| cognition | | | | |
| Number connection-A (sec) | 0.68 | 0.001 | 0.63 | 0.002 |
| Number connection-B (sec) | 0.445 | 0.04 | 0.28 | 0.24 |
| Serial dotting (sec) | 0.52 | 0.018 | 0.41 | 0.05 |
| Line drawing errors | 0.58 | 0.009 | 0.59 | 0.008 |
| (number) | | | | |
| Line drawing time (sec) | 0.24 | 0.31 | 0.17 | 0.48 |
| ICT lures (number) | 0.26 | 0.26 | 0.29 | 0.19 |
| Lower value | | | | |
| indicates poor cognition | | | | |
| Digit symbol (score) | −0.63 | 0.003 | −0.46 | 0.04 |
| Block design (score) | −0.27 | 0.25 | −0.21 | 0.37 |
| ICT targets (%) | −0.51 | 0.019 | −0.57 | 0.007 |

A high score on digit symbol, block design and ICT targets indicates good cognition; rest of the tests a high score indicates poor cognitive performance. Significant correlations are in bold text. Therefore we found a significant correlation between impairment on most cognitive tests and relative abundance of Alcaligeneceae and Porphyromonadaceae. All values are presented as mean±standard deviation unless mentioned otherwise.

Multivariate Analysis of the HE and no-HE groups: HE patients, as expected had a higher MELD score and bowel movement frequency compared to those without HE (Table 1). The rate of proton pump inhibitor use and other complications of cirrhosis were not different between the groups. Although the major families were present in both sample classes, there were observable abundance differences in some of the taxa; there was a significantly higher abundance of Veillonellaceae in the HE group (14±12% vs 4±9%, p=0.046) compared to the no HE group. There were no significant differences in the other microbiome families between the HE and no HE groups; Alcaligenaceae (11±12% vs. 13±14%, p=0.72), Bacteroidaceae (4.8±3.5% vs. 6.6±2.3%, p=0.18), Enterobacteriaceae (23±37% vs. 11±16%, p=0.285), Fusobacteriaceae (4.1±10.1% vs. 6.6% vs. 17.1%, p=0.72), Lachnospiraceae (24±26% vs. 41±18%, p=0.10), Lactobacillaceae (1±1% vs. 2±1%, p=0.23), Porphyromonadaceae (13±18% vs. 9±8%, p=0.53), Prevotellaceae (14±28% vs.19±34%, p=0.34), Ruminococcaceae (22±18% vs. 30±30%, p=0.52) and Streptococcaceae (3±8% vs. 1±1%, p=0.10).The MANOVA performed using Veillonellaceae, IL-13, IL-6, MELD score, and endotoxin demonstrated a p value of 0.002 using the Lawley-Hotelling test statistic of 2.25672 with an F statistic of 5.481.

Comparison within the HE group: There was no significant difference between the clinical, inflammatory or cognitive profile between HE patients on lactulose alone compared to those on lactulose and rifaximin (Table 6). Additionally, no differences in the microbiome components were identified using classic multivariate analysis. Specifically the normalized abundances at the family level of Alcaligenaceae (12.6±9% vs. 10±13%, p=0.8), Enterobacteriaceae (26±40% vs. 24±40%, p=0.7), Bacteroidaceae (39±35% vs. 58±39%, p=0.35), Porphyromonadaceae (14±21% vs. 8±15%, p=0.23), Prevotellaceae (18±25% vs. 10±19%, p=0.45), Veillonellaceae (15±13% vs 15±17%, p=0.8), Ruminococcaceae (17±22% vs. 17±19%, p=0.6), Streptococcaceae (4±10 vs. 2±3, p=0.55) and Lactobacillaceae (2±1% vs 1±1%, p=0.42) between the two groups were not statistically significant.

TABLE 6
Comparison within the HE group

| | On lactulose | On lactulose | |
|---|---|---|---|
| | alone | and rifaximin | P |
| | (n = 11) | (n = 6) | value |
| MELD score | 16.5 ± 7.6 | 18.2 ± 3.3 | 0.53 |
| Venous Ammonia | 52.8 ± 26.3 | 36.3 ± 32.6 | 0.23 |
| Endotoxin | 0.21 ± 0.21 | 0.41 ± 0.26 | 0.13 |
| IL-6 (pg/ml) | 47.8 ± 56.4 | 108.0 ± 94.3 | 0.20 |
| IL-2 (pg/ml) | 61 ± 108 | 21.6 ± 22.4 | 0.25 |
| TNF-α (pg/ml) | 6.7 ± 4.2 | 7.7 ± 4.22 | 0.67 |
| IL-13 (pg/ml) | 31.9 ± 84.2 | 32.1 ± 49.7 | 0.99 |
| Number connection-A (seconds) | 55.2 ± 34.1 | 79.4 ± 49.9 | 0.27 |
| Number connection-B (seconds) | 189 ± 127 | 231 ± 105 | 0.51 |
| Digit symbol (score) | 36.5 ± 14.0 | 34.8 ± 12.3 | 0.82 |
| Block design (score) | 24.9 ± 24.8 | 60.0 ± 53.4 | 0.23 |
| Serial dotting (seconds) | 88.7 ± 29.2 | 124.2 ± 36.5 | 0.10 |
| Line drawing errors (score) | 28.0 ± 18.6 | 43.0 ± 42.8 | 0.49 |
| Line drawing time (seconds) | 106.9 ± 54.5 | 79.4 ± 72.1 | 0.48 |
| ICT targets (%) | 90.6 ± 8.64 | 83.7 ± 21.0 | 0.52 |
| ICT lures (number) | 17.4 ± 10.8 | 19.6 ± 10.6 | 0.71 |

There was no significant difference in any variable tested between HE patients on lactulose alone compared to those on lactulose and rifaximin. All values are presented as mean±standard deviation unless mentioned otherwise.

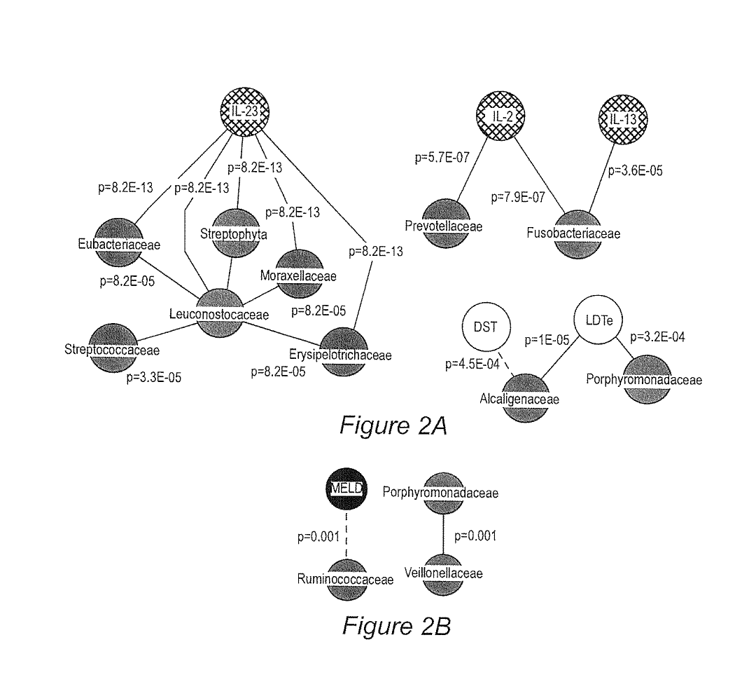

Correlation network analysis: Patients with HE: In contrast to the multivariate analysis above, several significantly strong correlations were found between features within the HE group with the correlation coefficients (FIG. 2A). IL-23 was an important correlate with several bacterial families across different phyla, such as Leuconostocaceae, Eubacteriaceae, Erysipelotrichaceae, Moraxellaceae, Streptophyta and Streptococcaceae within the HE group. All p-values for this correlation were below 8.2E-05 indicating a highly robust linkage. The correlation of immune function with bacterial families was further illustrated by the highly significant correlation (p values <3.5E-0.5) between inflammatory cytokines IL-2 and IL-13 with Fusobacteriaceae and Prevotellaceae. The correlation between Porphyromonadacae and Alcaligenacae with poor performance on cognitive tests was observed in this group accompanied by very significant p values (p<1E-05). Patients without HE: In sharp contrast, relatively few correlations that reached the stringent threshold we had set for this analysis were seen in patients without HE and markers of inflammation, cognition and microbial families. MELD score was negatively correlated with Ruminococcaceae while there was positive correlation between Porphyromonadaceae and Veillonellaceae (FIG. 2B). We did not find any significant correlations between inflammation and cognitive function that were abundant in the HE group correlation network.

Prospective study after lactulose withdrawal: Seven male cirrhotic patients with HE (age 53±8 years) controlled on lactulose underwent a systematic withdrawal of lactulose. All patients had alcoholic liver disease while five also had chronic hepatitis C. None of the patients had clinically recurrent HE at day 14. A significant (>1%) abundance was present for only 13 taxa at baseline on lactulose in those seven patients. These were mainly from the phylum Bacterioidetes (Bacteroides 35%, Prevotella 13%, Hallella 4%, Alistipes 3%, Parabacteroides spp. 2%, Porphyromonadaceae 1.7%) and Firmicutes (Faecalibacterium 7%, Lachnospira 5%, Roseburia 3%, Veillonella 2%, Dialister 2% and Succinispira spp. 2%) with little contribution of Proteobacteria (Alcaligenaceae 2.6%, Hafnia 2% and Sutterella spp. 1%) and none from Actinobacter spp. or Fusobacteria. There was <1% abundance on Lactobacillus spp., Clostridium spp., Streptococcus spp., Shigella spp. and Ruminococcus spp. After lactulose withdrawal, Faecalibacterium spp. (abundance on lactulose 6% to 1% post-withdrawal, p=0.026) and a trend towards Veillonella spp. (2% to 0%, p=0.07) appeared to decrease as a result of withdrawal. No other significant relative abundance change was identified, including Porphyroinonadacae and Alcaligenacae.

Discussion: This study demonstrates that a systems biology approach (correlation network analysis) can be used to identify key linkages between the microbiome, inflammatory milieu, endotoxemia, and cognition in patients with HE. The IL-23 system was highly correlated with several bacterial families in patients within the HE group and there was a direct correlation between cognition, Porphyromonadaceae and Alcaligeneceae. We found significant differences between the microbial flora of age-matched healthy controls to the cirrhotic population with a higher degree of difference in HE patients. The study showed that there was no significant difference in the stool flora between HE patient on lactulose compared to those additionally on rifaximin. The results also indicate that a systematic withdrawal of lactulose therapy had minimal effect on the gut microflora abundance.

To date, it has been difficult to identify significant differences between control and disease groups using classic multivariate approaches (27). Specifically, the composition of the human gut microbiome has been shown to vary significantly between individuals and this is a fundamental problem in associating the microbiome with diseases (12). Furthermore, most microbial abundance matrices derived from sequence data are both sparse and non-parametric. A microbial ecological interpretation of these issues is that different phylogenetic taxa play the same functional role in the complex non-linear interactions between the human host and gut microbiome. It should be noted that these interactions are not static but form a non-linear complex dynamic network that further confounds classic multivariate analysis methods. Unlike these methods, correlation network analysis allows the interrogation of these non-linear dynamics to correlate phylogenetically-defined taxa with function and disease phenotype. This was leveraged in our study where we found that HE was significantly correlated with microbiome components and inflammatory cytokines.

We found a significant difference in the bacterial composition of patients with cirrhosis compared to healthy controls that intensified when the cirrhotic group was divided into HE and those without HE. Rianinococeaceae and Clostridium incertae sedis XIV were over-represented in controls similar to prior studies in inflammatory bowel disease and cirrhosis (7, 18, 19, 25, 46). The findings are also similar to those published by Chen et al in cirrhotic patients despite differences in cirrhosis etiology and diet (7). However, their study did not evaluate HE specifically and they did not perform a systems biology analysis.

Alcaligenecaeae and Enterobacteriaceae were among the bacterial taxa that were differentially detected in cirrhotics with HE compared to controls but not different between controls and cirrhotics without HE. Increased Alcaligenaceae abundance was significantly associated with poor cognitive performance while Enterobacteriaceae were associated with worsening inflammation and MELD score in the cirrhosis group. The correlation between the MELD score, HE and Enterobacteriaceae accords with the observation that these bacteria are responsible for most of the life-threatening infections associated with advanced cirrhosis (35, 37). Also, the negative correlation of Ruminococcaceae with endotoxemia and MELD score and reduction in this class in cirrhotics overall could indicate a protective role.

The striking finding was the direct correlation between specific bacterial taxa and cognitive function. To our knowledge bacterial taxa have not been previously related to cognitive and inflammatory markers in cirrhosis using culture-independent techniques. Porphyromonadaceae and Alcaligeneceae were associated with poor cognition in almost all tests. It is unlikely that this is merely a manifestation of worsening liver disease because the MELD score was not significantly correlated either with cognitive performance or with these bacteria. Alcaligeneceae are Proteobacteria that are typically associated with opportunistic infections; interestingly they degrade urea to produce ammonia; which may explain part of this association (28). Porphyromonas are gram-negative anaerobes, whose fecal presence may be attributed to the deficient stomach acid and bile barrier function in cirrhosis (6, 33, 40). Interestingly, in animal studies, gut microbial colonization with specific bacteria has been shown to influence neuronal circuitry involved in motor control and behavior (9, 15). The correlation of these bacterial families with cognition in humans is a novel finding that needs further study.

We confirmed the pro-inflammatory milieu and endotoxemia in HE patients (36) and further demonstrated that specific microbial families, Enterobacteriacae, Veillonellaceae and Fusobacteriaceae were associated with inflammation (44). Specifically, in HE patients, inflammatory markers IL-23, IL-1b, IL-2, IL-4 and IL-13 were highly correlated with gut microbiome components, possibly indicating a synergy between inflammation and cognition with microbiome changes (20, 26). It is interesting that IL-13, which in addition to being an inflammatory mediator with IL-4 also mediates allergic reactions, would be increased in cirrhotic patients with HE. While none of our patients had an allergic diatheses, the increased IL-13 concentration may also represent its profibrotic potential and the widespread immuno-modulatory disturbances that are prevalent in cirrhosis (24). We did not find a difference in the inflammatory cytokines across etiologies which is likely due to the limited sample size and patients with dual etiologies. The IL-23/IL-17 pathway is triggered by exposure to infectious agents in the intestine, which releases a cascade of pro-inflammatory cytokines (10). IL-23 functions as a stimulant of IL-17 production and its role in Inflammatory Bowel Disease has been well described (1, 16). The correlation between IL-23 and several bacterial families indicates that IL-23/IL-17 cytokine pathway may be an important mechanism behind intestinal inflammation in HE and cirrhosis.

Strengthening these correlations was the minimal effect that lactulose withdrawal had on the stool flora composition after 14 days; this replicates prior non-culture based experience with lactulose in healthy individuals (39). We did not replicate prior culture-based studies in which lactulose therapy resulted in higher lactobacillus or reduction in E. coli and Staphyloccoci after symbiotic supplementation (23, 31). Our results are probably different due to the increased depth of the interrogation of the microbial community by MTPS rather than culture methodology. It is however possible that lactulose may act through change in bacterial functionality rather than change in abundances which were measured in this study. These results suggest that these microbial abundances are reflective of HE and cirrhosis rather than just lactulose therapy.

Collectively our data indicate that the gut microbiome components are significantly different between healthy controls and cirrhotic patients, especially those with HE, and are directly correlated with cognition in cirrhosis. Additionally, markers of the Th17 and innate immune response were significantly correlated with Alcaligeneceae, Porphyromonadaceae and Enterobacteriaceae in patients with HE. The IL-17/IL-23 pathway forms a key inflammatory link in this association. As noted above, these findings are beneficially applied to designing novel hypothesis driven research and therapies such as targeted prebiotics and probiotics aimed at enhancing cognition through modulation of these microbiome components.

### REFERENCES

1. Ahern P P, Schiering C, Buonocore S, McGeachy M J, Cua D J, Maloy K J, and Powrie F. Immunity 33: 279-288, 2010.
2. Bajaj J S. Aliment Pharmacol Ther 31: 537-547, 2010.
3. Bajaj J S, Hafeezullah M, Franco J, Varma RR , Hoffmann R G, Knox J F, Hischke D, Hammeke T A, Pinkerton S D, and Saeian K. Gastroenterology 135: 1591-1600 e1591, 2008.
4. Bajaj J S, Heuman D M, Wade J B, Gibson D P, Saeian K, Wegelin J A, Hafeezullah M, Bell D E, Sterling R K, Stravitz R T, Fuchs M, Luketic V, and Sanyal A J. Gastroenterology 140: 478-487 e471, 2011.
5. Bajaj J S, Schubert C M, Heuman D M, Wade J B, Gibson D P, Topaz A, Saeian K, Hafeezullah M, Bell D E, Sterling R K, Stravitz R T, Luketic V, White M B, and Sanyal A J. Gastroenterology 138: 2332-2340, 2010.
6. Bajaj J S, Zadvornova Y, Heuman D M, Hafeezullah M, Hoffmann R G, Sanyal A J, and Saeian K. Am J Gastroenterol 104: 1130-1134, 2009.
7. Chen Y, Yang F, Lu H, Wang B, Chen Y, Lei D, Wang Y, Zhu B, and Li L. Hepatology 54: 562-572, 2011.
8. Cordoba J. J Hepatol, 2011.
9. Cryan J F and O'Mahony S M. Neurogastroenterol Motil 23: 187-192, 2011.
10. D'Elios M M, Del Prete G, and Amedei A. Expert Opin Ther Targets 14: 759-774, 2010.
11. Folstein M F, Folstein S E, and McHugh P R. J Psychiatr Res 12: 189-198, 1975.
12. Gillevet P, Sikaroodi M, Keshavarzian A, and Mutlu E A. Chem Biodivers 7: 1065-1075, 2010.
13. Hamady M and Knight R. Genome Research 19: 1141-1152, 2009.
14. Haussinger D and Schliess F. Gut 57: 1156-1165, 2008.
15. Heijtz R D, Wang S, Anuar F, Qian Y, Bjorkholm B, Samuelsson A, Hibberd M L, Forssberg H, and Pettersson S. Proc Natl Acad Sci USA, 2011.
16. Huber S and Flavell R A. Immunity 33: 150-152, 2010.
17. Jin J J, Kim H D, Maxwell J A, Li L, and Fukuchi K. J Neuroinflammation 5: 23, 2008.
18. Joossens M, Huys G, Cnockaert M, De Preter V, Verbeke K, Rutgeerts P, Vandamme P, and Vermeire S. Gut 60: 631-637, 2011.
19. Kang S, Denman S E, Morrison M, Yu Z, Dore J, Leclerc M, and McSweeney C S. Inflamm Bowel Dis.
20. Keshavarzian A, Holmes E W, Patel M, Iber F, Fields J Z, and Pethkar S. Leaky gut in alcoholic cirrhosis: a possible mechanism for alcohol-induced liver damage. Am J Gastroenterol 94: 200-207, 1999.
21. Komanduri S, Gillevet P M, Sikaroodi M, Mutlu E, and Keshavarzian A. Dysbiosis in pouchitis: evidence of unique microfloral patterns in pouch inflammation. Clin Gastroenterol Hepatol 5: 352-360, 2007.
22. Lane D J. 16s/23s rRNA sequencing. In: Nucleic acid techniques in bacterial systematics, edited by Goodfellow M. West Sussex, England: John Wiley & Sons Ltd, 1991, p. 115-175.
23. Liu Q, Duan Z P, Ha D K, Bengmark S, Kurtovic J, and Riordan S M. Hepatology 39: 1441-1449, 2004.
24. Marra F and Annunziato F. Gut 59: 868-869, 2010.
25. Mondot S, Kang S, Furet J P, Aguirre de Career D, McSweeney C, Morrison M, Marteau P, Dore J, and Leclerc M. Inflamm Bowel Dis.
26. Mutlu E, Keshavarzian A, Engen P, Forsyth C B, Sikaroodi M, and Gillevet P. Alcohol Clin Exp Res 33: 1836-1846, 2009.
27. Naqvi A, Rangwala H, Keshavarzian A, and Gillevet P. Chem Biodivers 7: 1040-1050, 2010.
28. Obata T, Goto Y, Kunisawa J, Sato S, Sakamoto M, Setoyama H, Matsuki T, Nonaka K, Shibata N, Gohda M, Kagiyama Y, Nochi T, Yuki Y, Fukuyama Y, Mukai A, Shinzaki S, Fujihashi K, Sasakawa C, Iijima H, Goto M, Umesaki Y, Benno Y, and Kiyono H. Proc Natl Acad Sci USA 107: 7419-7424, 2010.
29. Ridlon J M, McGarr S E, and Hylemon P B. Clin Chim Acta 357: 55-64, 2005.
30. Riggio O, Ridola L, Pasquale C, Nardelli S, Pentassuglio I, Moscucci F, and Merli M. Clin Gastroenterol Hepatol 9: 181-183, 2011.
31. Riggio O, Varriale M, Testore G P, Di Rosa R, Di Rosa E, Merli M, Romiti A, Candiani C, and Capocaccia L. J Clin Gastroenterol 12: 433-436, 1990.
32. Romero Gomez M, Cordoba J, Jover R, del Olmo J, Fernandez A, Flavia M, Company L, Poveda M J, and Felipo V. Med Clin (Bare) 127: 246-249, 2006.
33. Savarino V, Mela G S, Zentilin P, Mansi C, Mele M R, Vigneri S, Cutela P, Vassallo A, Dallorto E, and Celle G. J Hepatol 25: 152-157, 1996.
34. Shannon P, Markiel A, Ozier O, Baliga N S, Wang J T, Ramage D, Amin N, Schwikowski B, and Ideker T. Genome Res 13: 2498-2504, 2003.
35. Shawcross D L, Sharifi Y, Canavan J B, Yeoman A D, Abeles R D, Taylor N J, Auzinger G, Bernal W, and Wendon J A. J Hepatol 54: 640-649, 2011.
36. Shawcross D L, Wright G, Olde Damink S W, and Jalan R. Role of ammonia and inflammation in minimal hepatic encephalopathy. Metab Brain Dis 22: 125-138, 2007.
37. Tandon P and Garcia-Tsao G. Semin Liver Dis 28: 26-42, 2008.
38. Turnbaugh P J, Hamady M, Yatsunenko T, Cantarel B L, Duncan A, Ley R E, Sogin M L, Jones W J, Roe B A, Affourtit J P, Egholm M, Henrissat B, Heath A C, Knight R, and Gordon J I. Nature 457: 480-484, 2009.
39. Vanhoutte T, De Preter V, De Brandt E, Verbeke K, Swings J, and Huys G. Appl Environ Microbiol 72: 5990-5997, 2006.
40. Vlahcevic Z R, Buhac I, Bell C C, Jr., and Swell L. Gut 11: 420-422, 1970.
41. Weissenborn K, Ennen J C, Schomerus H, Ruckert N, and Hecker H. J Hepatol 34: 768-773, 2001.
42. White J R, Nagarajan N, and Pop M. PLoS Comput Biol 5: e1000352, 2009.
43. Wong F, Bernardi M, Balk R, Christman B, Moreau R, Garcia-Tsao G, Patch D, Soriano G, Hoefs J, and Navasa M. Sepsis in cirrhosis: report on the 7th meeting of the International Ascites Club. Gut 54: 718-725, 2005.
44. Wright G and Jalan R. Hepatology 46: 291-294, 2007.
45. Wu S, Yin R, Ernest R, Li Y, Zhelyabovska O, Luo J, Yang Y, and Yang Q. Cardiovasc Res 84: 119-126, 2009.
46. Zoetendal E G, Ben-Amor K, Harmsen H J, Schut F, Akkermans A D, and de Vas W M. Appl Environ Microbiol 68: 4225-4232, 2002.

### Example 2

### The Colonic Mucosal Microbiome Differs from Stool Microbiome in Cirrhosis and Hepatic Encephalopathy and is Linked to Cognition and Inflammation

### Introduction

The pathogenesis of cirrhosis and its complications, specifically bacterial translocation, infections and hepatic encephalopathy are closely related with changes in the intestinal microflora (39, 43). Recent studies have demonstrated differences in the stool microbiome of patients with cirrhosis compared to healthy individuals, especially regarding the presence of resident or autochthonous bacteria (7, 10). However, despite significant differences in the clinical, pro-inflammatory milieu and cognitive function, in some respects there was minimal difference in stool microbiome between cirrhotics with hepatic encephalopathy (HE) and those without (No-HE) (7, 35, 36). This was intriguing since HE therapies are hypothesized to act by influencing the gut bacteria (11). Studies in non-cirrhotic populations have demonstrated changes in the intestinal mucosa microbiome compared to the stool but this has not been studied in cirrhosis to date (17, 46).

The aim of the study was to evaluate changes between the stool and colonic mucosal microbiome of cirrhotic patients with and without HE and to link them with changes in peripheral inflammation and cognition. The a priori hypothesis was that there would be a significant difference in the microbiome composition of the colonic mucosa compared to the stool in cirrhotic patients with HE compared to those without HE and that these shifts in the mucosal microbiome would be associated with changes in inflammation and cognition.

### Materials and Methods

Patients with cirrhosis with or without HE were included for a one-time visit. We excluded patients with a current infection (defined by elevated white blood cell (WBC) count, clinical suspicion or fever), variceal bleeding within the last 4 weeks, on gut-absorbable antibiotics, or had alcohol or illicit drug intake within 3 months (checked by drug and alcohol screens). Patients in the “No-HE” group had never had an episode of HE and were not on any therapy for it. Patients in the “HE” group had suffered at least one HE episode within the last 3 months and were currently controlled on lactulose alone or lactulose with rifaximin.

During the visit, the subjects underwent a physical examination and measurement of body mass index (BMI), detailed analysis of medical records including current medications, a detailed dietary recall and collection of stool and blood samples. All subjects underwent a mini-mental status exam and only those scoring above 25 were included in the full study (14). Subsequently a recommended cognitive battery consisting of the following tests was administered; (a) Psychometric hepatic encephalopathy score (PHES), (b) block design test (subjects are required to replicate designs with given blocks in a timed manner) and (c) Inhibitory control test [ICT: Subjects are instructed to respond to alternating presentations of X and Y on the screen (targets) while inhibiting response when X and Y are not alternating (lures)] (5, 41). The PHES consists of number connection test-A/B (subjects are asked to “join the dots” between numbers or numbers and alphabets in a timed fashion), digit symbol (subjects are required to copy corresponding figures from a given list within 2 minutes), line drawing (time) and (errors): subjects are required to trace a line between two parallel lines and balance between speed and accuracy. Time required and the number of times the subject's line strays beyond the marked lines (errors) are recorded] and serial dotting (subjects are asked to dot the center of a group of blank circles)]. The PHES is a validated battery for cognitive dysfunction in cirrhosis and tests for psychomotor speed, visuo-motor coordination, attention and set-shifting(32). Block design tests for visuo-motor coordination. A high score on block, digit symbol and ICT targets and a low score on the rest indicate good performance.

Blood was collected for evaluation of venous ammonia, MELD score and inflammatory cytokines. A portion of the serum was stored at −80° C., which was subsequently analyzed for innate immunity [IL-1b, IL-6, TNF-α], Th1 ([IFN-γ (interferon-gamma) and IL-2], Th2 (IL-4, IL-10, IL-13] and Th17 responses (IL-17 and IL-23), endotoxin, neural function [neuron-specific enolase (NSE) and s100b protein](44), endothelial activation [soluble intravascular adhesion molecule (sICAM-1) and soluble vascular adhesion molecule (sVCAM-1)] and asymmetric di-methyl arginine (ADMA). These were analyzed in duplicate using published techniques by AssayGate Inc, Ijamsville, Md. (6, 7).

Interrogation of the Microbiome: Stool was collected and DNA extracted for microbiome analysis using published techniques (30). A subset underwent an un-sedated, unprepared flexible sigmoidoscopy during which a pinch biopsy of the recto-sigmoid mucosa was obtained, which was snap-frozen and stored at −80° C. till the analysis. We first use Length Heterogeneity PCR (LH-PCR) fingerprinting of the 16S rRNA to rapidly survey samples and standardize the community amplification. We then interrogated the microbial taxa associated with the gut fecal microbiome using Multitag Pyrosequencing (MTPS) (16). This technique allows for rapid sequencing of multiple samples at one time yielding thousands of sequence reads per sample.

Microbiome Community Fingerprinting: LH-PCR was done to standardize the community analysis as previously published. Briefly, total genomic DNA was extracted from tissue using Bio101 kit from MP Biomedicals Inc., Montreal, Quebec as per the manufacturer's instructions. About 10 ng of extracted DNA was amplified by PCR using a fluorescently labeled forward primer 27F (5′-(6FAM) AGAGTTTGATCCTGGCTCA G-3′, SEQ ID NO: 1) and unlabeled reverse primer 355R′ (5′-GCTGCCTCCCGTAGGAGT-3′, SEQ ID NO: 2). Both primers are universal primers for Bacteria (23). The LH-PCR products were diluted according to their intensity on agarose gel electrophoresis and mixed with ILS-600 size standards (Promega) and HiDi Formamide (Applied Biosystems, Foster City, Calif.). The diluted samples were then separated on a ABI 3130x1 fluorescent capillary sequencer (Applied Biosystems, Foster City, Calif.) and processed using the Genemapper™ software package (Applied Biosystems, Foster City, Calif.). Normalized peak areas were calculated using a custom PERL script and operational taxonomic units (OTUs) constituting less than 1% of the total community from each sample were eliminated from the analysis to remove the variable low abundance components within the communities.

MTPS: We employed the MTPS process to characterize the microbiome from the fecal and biopsy samples. Specifically, we have generated a set of 96 emulsion PCR fusion primers that contain the 454 emulsion PCR linkers on the 27F and 355R primers and a different 8 base “barcode” between the A adapter and 27F primer. Thus, each fecal sample was amplified with unique bar-coded forward 16S rRNA primers and then up to 96 samples were pooled and subjected to emulsion PCR and pyrosequenced using a GS-FLX pyrosequencer (Roche). Data from each pooled sample were “deconvoluted” by sorting the sequences into bins based on the barcodes using custom PERL scripts. Thus, we were able to normalize each sample by the total number of reads from each barcode. We have noted that ligating tagged primers to PCR amplicons distorts the abundances of the communities and thus it is critical to incorporate the tags during the original amplification step.

Microbiome Community Analysis: We identified the taxa present in each sample using the Bayesian analysis tool in Version 10 of the Ribosomal Database Project (RDP10). The abundances of the bacterial identifications were then normalized using a custom PERL script and genera present at >1% of the community were tabulated. We chose this cutoff because of our a priori assumption that genera present in <1% of the community vary between individuals and have minimal contribution to the functionality of that community and 2,000 reads per sample will only reliably identify community components that are greater than 1% in abundance (16).

Statistical analysis: Cirrhotics with HE were compared to those without HE with respect to BMI, inflammatory markers, cognitive performance and microbiome constituents. Unpaired t-tests were used to compare demographics, cognitive tests and inflammatory markers. Since the microbiome constituents tend to be sparse and non-parametrically distributed, we used Metastats to compare microbiome between stool and mucosa of patients with and without HE (42). Metastats performs statistical analysis (to investigate metagenomic differences) along with biomarker discovery (to evaluate specific features underlying these differences) based on repeated t statistics and Fisher's tests on random permutations (34). We also performed Metastats analysis between the mucosal microbiome compared to the stool microbiome in patients with HE and without HE and those in HE on or not on rifaximin. Principal Component Analysis (PCO) on the abundance tables and weighted and unweighted UNIFRAC analysis using the QIIME pipeline were also performed (22, 25). Subsequently, we analyzed the correlations between MELD score, BMI, inflammatory markers, cognitive tests and microbiome constituents using a correlation network analysis obtained through a customized statistical script in R (7) using a P-value cutoff of <0.05 and an R value >0.5 to identify the most significant relationships (4, 16).

### Results:

A total of 60 patients with cirrhosis were included in the study. The distribution of HE and No-HE was relatively uniform with 24 patients without HE and 36 with HE. Of the 36 HE patients, 17 were only on lactulose while 19 were on both lactulose and rifaximin therapy. All patients were non-vegetarians and had similar dietary intake and constituents on recall prior to sample collection (mean intake 2350 Kcal and 14% protein intake). HE patients had a significantly higher MELD score and also, as expected, higher ammonia and worse cognitive performance on all tests compared to patients without HE (Table 7). There was higher endotoxin, s100b, IL-6 and ADMA in the HE patient group. Of the 60 patients, 36 (17 patients without HE and 19 with HE) underwent flexible sigmoidoscopy with biopsy the same day of the stool and sample collection. Patients on rifaximin had a worse cognitive performance compared to those only on lactulose on number connection-A (68.3 vs. 52.25 seconds) and B (192.9 vs. 145.8 seconds), targets (86 vs. 90%), serial dotting (94.2 vs. 84.0 seconds), line tracing errors (62.5 vs. 44.9) but not line tracing time (118.0 vs. 127.3), digit symbol (37 vs. 38 score), block design (17.5 vs. 19.8 score), and lures (15.7 vs 16.9 responses). Rifaximin-treated patients also had a significantly higher level of IL-6 (51.04 vs. 30.13) and endotoxin (0.43 vs. 0.20), with a trend towards higher MELD (18 vs 16) compared to those on lactulose alone.

TABLE 7
Comparison of Clinical parameters between Patients with and without HE

| | Cirrhosis without | Cirrhosis with |
|---|---|---|
| | HE (n = 24) | HE (n = 36) |
| Age | 54 ± 6 | 56 ± 4 |
| Gender (Male/Female) | 20/4 | 30/6 |
| Body Mass Index | 28.9 ± 4.4 | 29.0 ± 6.7 |
| MELD score | 10.4 ± 4.1 | 17.3 ± 6.8* |
| Venous Ammonia | 32.8 ± 12.6 | 48.8 ± 27.5* |
| IL-1b (pg/m1) | 14.3 ± 58.7 | 6.2 ± 12.1 |
| IL-2(pg/ml) | 15.0 ± 62.3 | 24.4 ± 68.6 |
| Interferon-gamma (pg/ml) | 10.1 ± 33.1 | 15.9 ± 54.5 |
| TNF-alpha (pg/ml) | 13.9 ± 43.2 | 7.4 ± 8.2 |
| IL-4 (pg/ml) | 16.3 ± 55.5 | 25.3 ± 94.5 |
| IL-6 (pg/ml) | 12.2 ± 32.5 | 40.6 ± 63.3* |
| IL-10 (pg/ml) | 10.4 ± 28.3 | 4.88 ± 5.89 |
| ADMA (gm/ml) | 0.38 ± 0.13 | 0.558 ± 0.20* |
| S100b protein (pg/ml) | 34.7 ± 27.4 | 57.1 ± 49.4* |
| Endotoxin (EU/ml) | 0.06 ± 0.01 | 0.32 ± 0.26* |
| Neuron-specific | 7461 ± 4288 | 6793 ± 3424 |
| enolase (pg/ml) | | |
| IL-23 (pg/ml) | 519 ± 1137 | 1130 ± 3289 |
| IL-17 (pg/ml) | 5.9 ± 16.9 | 17.8 ± 60.4 |
| sVCAM-1 (pg/ml) | 1488817 ± 664793 | 1683186 ± 794252 |
| sICAM-1 (pg/ml) | 319844 ± 268066 | 304680 ± 214037 |
| Number connection-A (sec) | 35.0 ± 14.7 | 60.3 ± 41.4* |
| Number connection-B (sec) | 95.9 ± 49.2 | 170 ± 123* |
| Digit Symbol (raw score) | 57.7 ± 12.0 | 37.4 ± 15.8* |
| Block Design (raw score) | 30.1 ± 14.9 | 18.7 ± 16.7* |
| Lures (number incorrect) | 10.5 ± 7.7 | 16.4 ± 10.3* |
| Targets (% correct) | 95.3 ± 8.7 | 88.1 ± 13.7* |
| Serial Dotting (sec) | 64.6 ± 18.7 | 89.3 ± 34.9* |
| Line Tracing time (sec) | 95.0 ± 37.7 | 122.5 ± 56.9* |
| Line Tracing errors (number) | 28.2 ± 20.4 | 54.0 ± 39.1* |

As expected patients with HE have a worse MELD score, cognitive performance and higher venous ammonia, endotoxin, IL-6, ADMA and S100b protein compared to patients without HE. A high score in Digit symbol, Block design and Targets indicates good cognitive performance while a high score in the remaining cognitive tests suggests poor performance. ADMA: asymmetric di-methyl arginine, sICAM-1: soluble intravascular adhesion molecule, sVCAM-1: soluble vascular adhesion molecule. All data is presented as mean+standard deviation, *=p<0.05

Comparison between all patients' mucosa to stool microbiome: We found a significant change in the microbiome of the mucosa compared to stool in the entire group using Metastats (Table 8). This change persisted when the comparison between stool and mucosa was performed for the HE and the no-HE group. The composition of the mucosal microbiome in the entire population differed considerably from the corresponding stool microbiome. Prominent bacterial genera found at a higher abundance in the mucosa belonged to Firmicutes (Blautia, Incertae Sedis XI), Actinobacteria (Propionibacterium and Streptomyces) and Proteobacteria (Vibrio). Interestingly, most bacteria found in higher abundances in stool were Firmicutes (Leuconostoc, Roseburia, Veillonella and Incertae Sedis XIV). These differences persisted when the group was divided into HE and no-HE (Tables 9 and 10). Propionibacterium and Vibrio genera were significantly more abundant in the mucosa than in the stool in both HE and no-HE.

TABLE 8
Cirrhosis mucosa vs cirrhosis stool comparison using Metastats

| Family_Genus (% abundance) | Mucosa | Stool | P value |
|---|---|---|---|
| Incertae Sedis XIV_Blautia | 4.1 | 1.5 | 0.002 |
| Vibrionaceae_Vibrio | 3.1 | 0.0 | 0.001 |
| Propionibacteriaceae_Propionibacterium | 1.3 | 0.0 | 0.001 |
| Streptomycetaceae_Streptomyces | 1.5 | 0.0 | 0.001 |
| Incertae Sedis XI_other | 0.5 | 0.0 | 0.001 |
| Incertae Sedis XIV_other | 0.3 | 1.2 | 0.005 |
| Veillonellaceae_Veillonella | 0.2 | 2.2 | 0.01 |
| Leuconostocaceae_Leuconostoc | 0.0 | 1.0 | 0.001 |
| Bacteroidales_incertae_sedis_other | 0.0 | 0.2 | 0.001 |
| Lachnospiraceae_Roseburia | 0.2 | 1.0 | 0.03 |

There was a significantly higher Blautia, Vibrio, Incertae Sedis XI, Propionibacterium and Streptomyces abundance and lower Incertae Sedia XIV, Veillonella, Bacteroides and Roseburia in the stool.

TABLE 9
HE mucosa vs HE stool

| Family_Genus (% abundance) | HE mucosa | HE stool | P value |
|---|---|---|---|
| Incertae Sedis XIV_Blautia | 5.2 | 1.6 | 0.004 |
| Vibrionaceae_Vibrio | 4.4 | 0 | 0.01 |
| Propionibacteriaceae_ | 1.1 | 0 | 0.001 |
| Propionibacterium | | | |
| Incertae Sedis XI_other | 0.8 | 0 | 0.001 |
| Vibrionaceae_other | 0.6 | 0 | 0.001 |
| Incertae Sedis XIV_other | 0.3 | 1.4 | 0.005 |
| Fusobacteriaceae_other | 0 | 1.1 | 0.001 |

TABLE 10
No-HE mucosa vs No-HE stool

| | No-HE | No-HE | |
|---|---|---|---|
| Family_Genus (% abundance) | mucosa | stool | P value |
| Leuconostocaceac_Leuconostoc | 0 | 1.0 | 0.002 |
| Bacteroidales_incertae_sedis_other | 0 | 1.0 | 0.001 |
| Alcaligenaceae_other | 0 | 0.8 | 0.001 |
| Streptomycetaceae_Streptomyces | 2.5 | 0 | 0.001 |
| Propionibacteriaceae_ | 1.8 | 0 | 0.001 |
| Propionibacterium | | | |
| Vibrionaceae_Vibrio | 1.3 | 0 | 0.001 |
| Burkholderiaceae_Ralstonia | 0.5 | 0 | 0.001 |

Comparison between HE and No-HE patients' microbiome: Next we compared the stool and mucosal microbiome of the HE and No-HE groups. We again found no appreciable difference in the stool microbiome between patients with and without HE despite the higher sample size in this study. However there was a significant difference in the mucosal microbiome between HE and no-HE patients (Table 11). Specifically, Firmicutes such as members of genera Veillonella, Megasphaera, Bifidobacterium and Enterococcus were higher in HE while Roseburia was more abundant in the no-HE group. We did not see significant clustering of the disease classes (HE and no-HE) in this sample set using either PCO or UniFrac analysis (data not shown).

Comparison between patients on lactulose alone compared to those on lactulose and rifaximin: As found between HE and no-HE patients, there was no difference in the stool microbiome of patients on rifaximin and lactulose compared to those on lactulose alone. The mucosal microbiome in rifaximin-treated patients however was significantly different (Table 9). There was a significantly decreased abundance of autochthonous bacteria (Roseburia and Blautia) and Veillonellaceae but an increased abundance of Propionibacterium in the rifaximin group.

TABLE 11
Comparison between mucosal microbiome abundances between HE
and no-HE groups using Metastats.

| | HE | No-HE | |
|---|---|---|---|
| Family_Genus (% abundance) | mucosa | mucosa | P value |
| Lachnospiraceae_Roseburia | 0.5 | 2.5 | 0.002 |
| Veillonellaceae_Veillonella | 0.7 | 0 | 0.001 |
| Burkholderiaceae_other | 0.8 | 0 | 0.001 |
| Veillonellaceae_Megasphaera | 2.4 | 0 | 0.001 |
| Streptomycetaceae_Streptomyces | 2.7 | 0 | 0.001 |
| Fusobacteriaceae_other | 3.5 | 0 | 0.001 |
| Bifidobacteriaceae_Bifidobacterium | 3.8 | 0 | 0.001 |
| Enterococcaceae_Enterococcus | 7.7 | 0 | 0.001 |

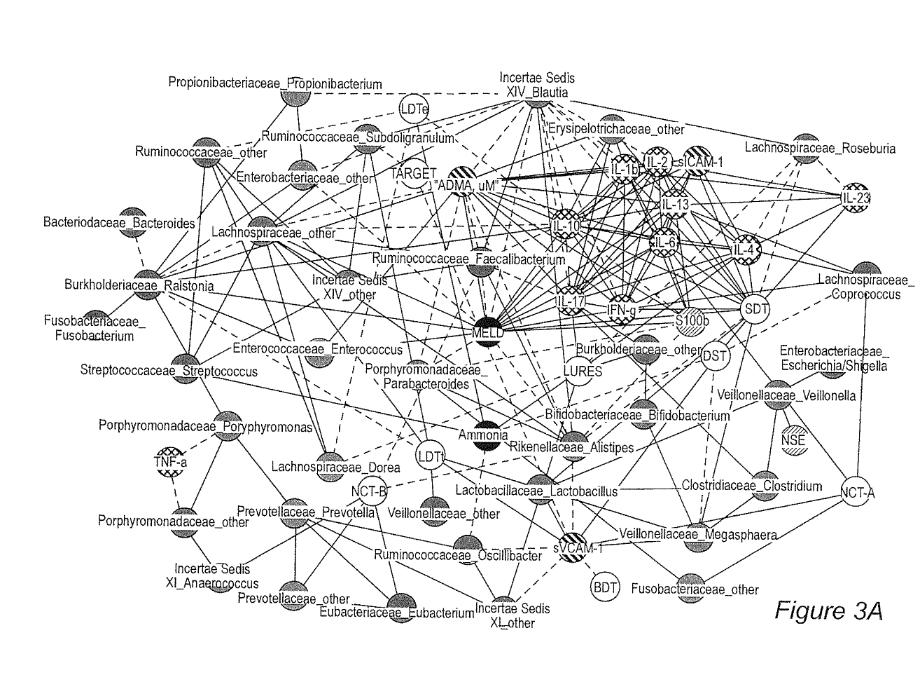

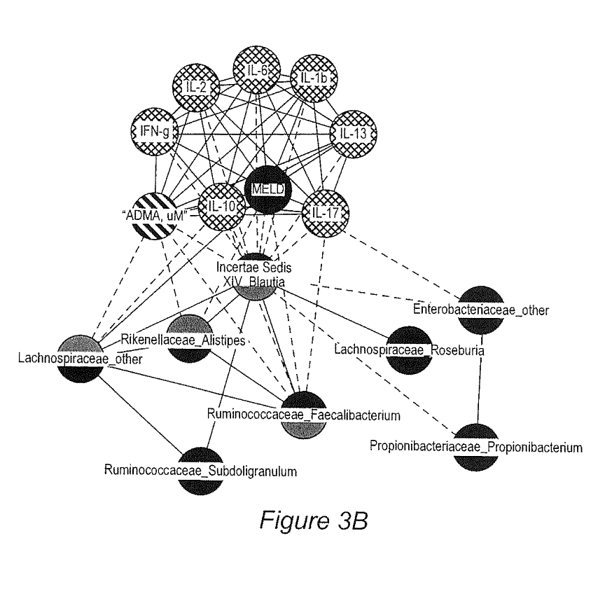

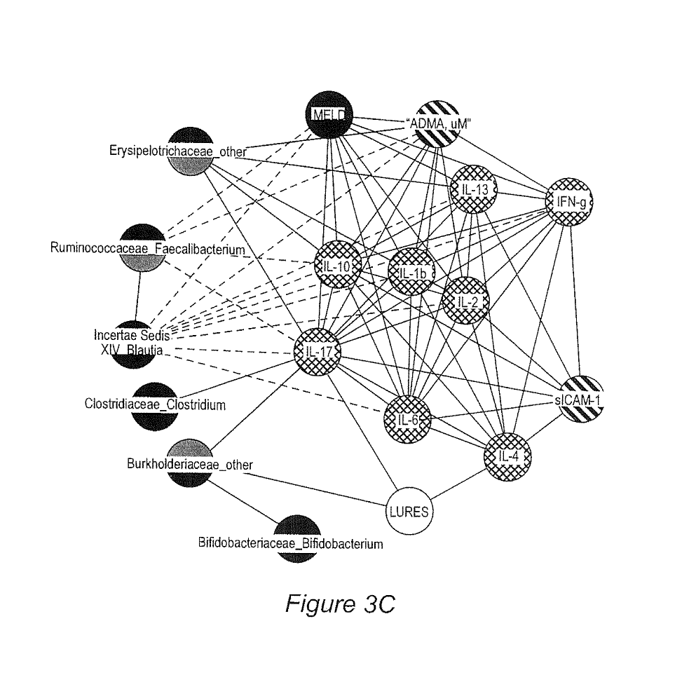

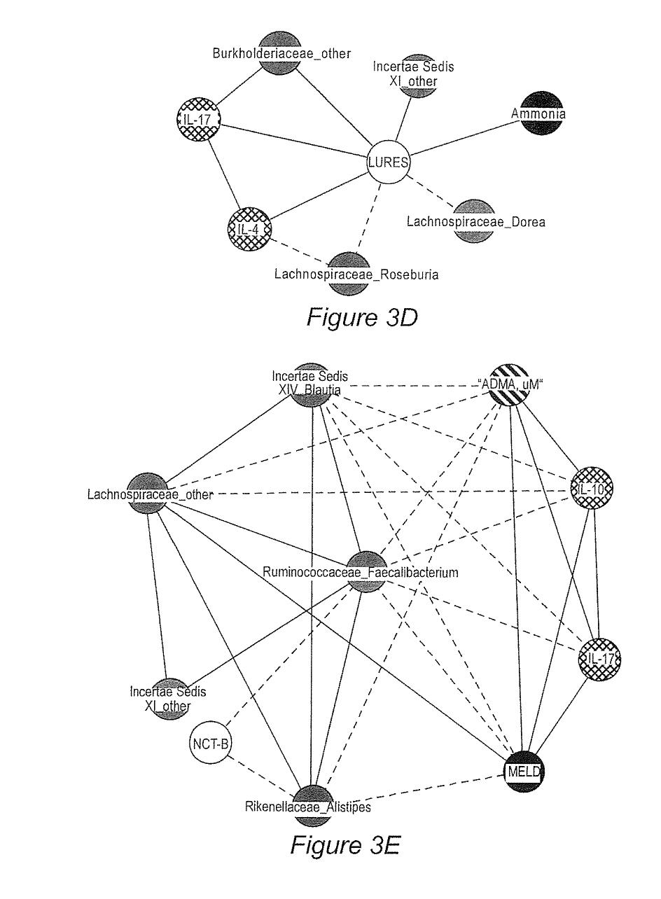

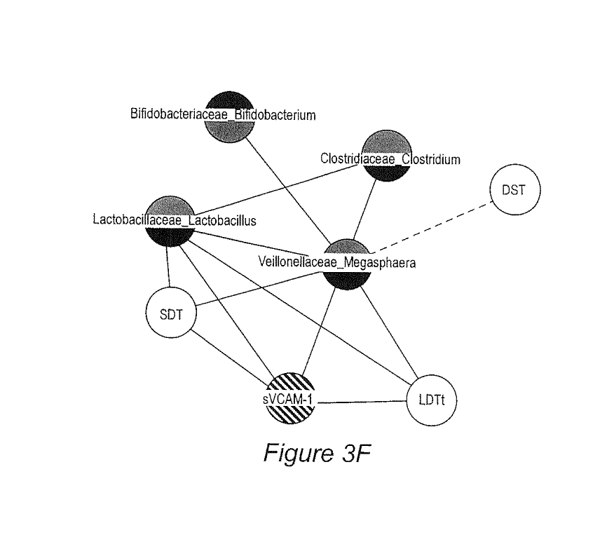

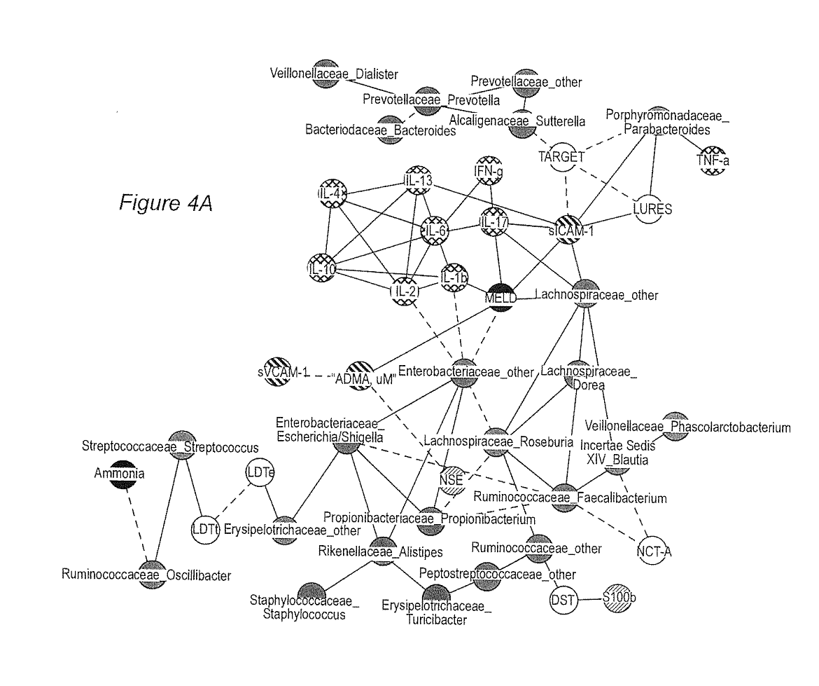

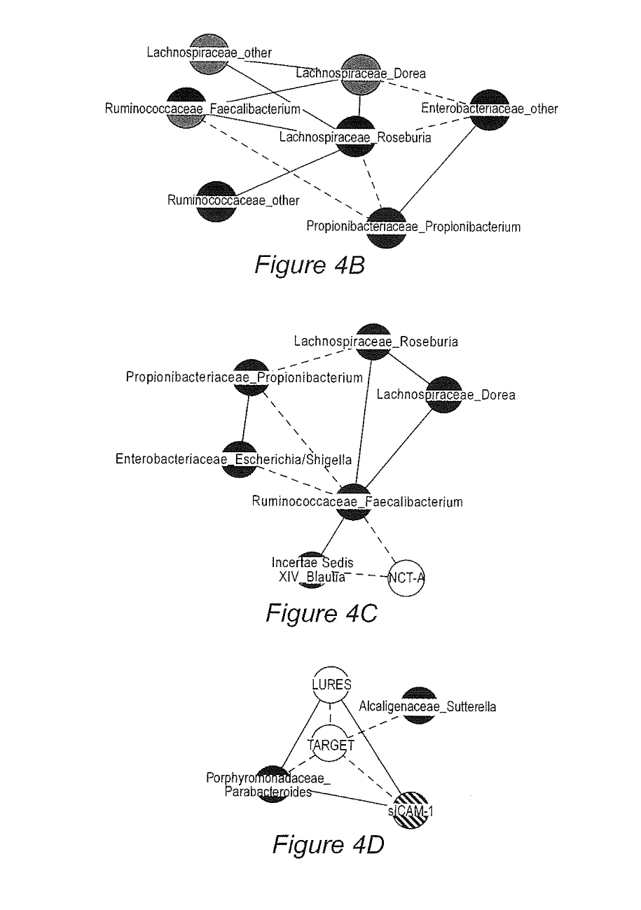

Correlation network analysis: We performed a Spearman correlation using a custom R package to analyze linkages between the cognitive performance and inflammatory markers and the mucosal microbiome in HE and No-HE patients. We did not perform the analysis with the stool microbiome since there was no significant difference between the two groups' stool microbiome using Metastats. The overall view of the two networks shows a distinct increase in the connectivity within the HE network (FIG. 3A) compared to the No-HE network (FIG. 4A). Certain bacterial genera were negatively correlated with inflammation and endothelial activation and linked to good cognitive performance across both networks. These were Fecalibacterium, Roseburia, other Lachnospiraceae and Blautia. We also found a significant dense correlation network surrounding IL-17 and MELD with other inflammatory markers and cognitive performance in both networks. Replicating our prior experience, we found members of the Alcaligeneceae and Porphyromonadaceae families associated with poor cognitive performance in the No-HE network.

What was interesting is that the genera present in higher abundance in the HE patients' mucosa (Table 12) was associated with higher inflammation, worse cognition and worse endothelial activation in the correlation network (FIG. 3A). Specifically, the sub-networks centered on Megaspheara, Veillonella, Burkholderia and Bifidobacterium showed that they were associated with poor cognition, higher MELD, higher inflammation and endothelial activation. These genera were not present in the no-HE network. In contrast, Roseburia, which was higher in the no-HE group, was associated with beneficial effects, i.e. less inflammation and endothelial activation and better cognition in both networks. FIGS. 3A and 4a are the correlation networks for the HE and no-HE groups' mucosal microbiome respectively. The figures that follow are sub-networks within both networks that show similar correlations between bacterial genera. FIG. 3B shows the node Incertae Sedis XIV_Blautia, FIG. 3C shows IL-17, FIG. 3D with Lures, FIG. 3E with Fecalibacterium and FIG. 3F with Megasphaera. FIGS. 4B through 4D display the sub-networks of the no-HE mucosal microbiome. FIG. 4B and 4C show connections Roseburia and Fecalibacterium respectively while FIG. 4D shows the connections between Lures and targets with bacterial genera.

TABLE 12
Comparison of the mucosal microbiome between patients on lactulose
alone compared to those on lactulose and rifaximin using Metastats

| | Lactulose | Rifaximin and | |
|---|---|---|---|
| Family_Genus (% abundance) | alone | lactulose | p-value |
| Incertae Sedis XIV_Blautia | 4.2 | 1.5 | 0.008 |
| Lachnospiraceae_Roseburia | 1.9 | 0 | 0.005 |
| Propionibacteriaceae_ | 1.1 | 2.3 | 0.03 |
| Propionibacterium | | | |
| Veillonellaceae_Other | 1.1 | 0 | 0.03 |
| Rikenellaceae_Alistipes | 1.8 | 0 | 0.03 |

DISCUSSION: We found a significant alteration in the colonic mucosal microbiome compared to stool in cirrhosis. We did not find any significant change in the stool microbiome between patients with or without HE but found a dramatic change in the mucosal microbiome between the two groups. However there was a difference between HE and no HE patients compared to healthy controls and stool microbiome was independently correlated with cognition, inflammation, endotoxemia and endothelial dysfunction. Specifically, we found a higher abundance of the beneficial genus Roseburia in patients without HE while a higher abundance of Enterococcus, Veillonella, Megasphaera, Bifidobacterium and Burkholderia was found in the HE patients' mucosa. The correlation network linking the mucosal microbiome to cognition, endothelial activation, inflammation and disease severity was richer in connectivity in the HE group. Bacterial genera such as Roseburia and Fecalibacterium were associated with better cognition, lower inflammation and endothelial activation in cirrhotics with and without HE. Genera that were over-represented in the HE mucosa, Enterococcus, Burkholderia, Megasphaera and Veillonella, were associated with worse cognition, inflammation and endothelial activation.

The differences between the mucosa and stool microbiome has been shown in several disease conditions such as Crohn's disease as well as in healthy volunteers (38). Prior studies have also shown that the influence of the fecal microbes may be less than that of the mucosal microbiome on immunity and overall health (17, 24). The intestinal barrier has a strong immunological interface comprised of mucus, epithelium and the mucosa-associated immune cells. The bacterial bio-film is usually restricted to the outer mucus layer (21, 27). However, there is evidence of cross-talk between the mucosal immune system and the gut bacterial species that can usually differentiate between commensals and pathogens (9). This study showed a significant difference between the mucosal and stool microbiome in the overall population and when divided into HE and no-HE. The study of the mucosal microbiome in cirrhosis is relevant because most pre-mortal events in cirrhosis, such as spontaneous bacterial peritonitis and spontaneous bacteremia, are related to intestinal bacterial translocation (2, 39). Alteration in permeability, bacterial overgrowth, poor motility, along with deficiency of anti-microbial peptides, further increases the risk of bacterial translocation in cirrhosis (33, 40, 43). The underlying suppression of the mucosal immunity in cirrhosis with the resultant pro-inflammatory milieu, leads to endotoxemia and complications of cirrhosis and HE (39, 43).

We confirmed our prior study demonstrating that there was no appreciable difference in the fecal bacterial composition of patients with and without HE, including those on rifaximin or not (7). However stool samples and mucosal samples independently correlate with inflammation, endotoxemia, endothelial function and cognitive dysfunction. Therefore even though there is no difference between the stools between patients with and without HE, they independently predict the outcomes. This was intriguing because patients with HE were significantly different from a liver disease, clinical severity, inflammatory and cognitive standpoint from those without HE. Therefore the changes in the mucosal bacterial composition were sought and were found to be significantly different between the groups. We found a higher abundance of autochthonous bacteria, Roseburia in the No-HE patients. Roseburia is one of the few genera that can produce butyrate, the preferred fuel source for colonocytes and is usually over-represented in healthy controls compared to any disease state (8). Therefore its higher abundance in the less affected group is consistent with previous findings. Autochthonous bacteria such as Roseburia have evolved to survive in the mucosal niches without eliciting a host immune reaction despite the abundant antimicrobial peptides (28). In contrast, genera such as Enterococcus are usually present in the fecal stream, not the mucosa (28). Interestingly we found an increase in abundance of potentially pathogenic genera, Enterococcus, Burkholderia and Veillonellaceae constituents, in HE patients. Prior stool studies have shown an increased abundance of Veillonellaceae in cirrhosis compared to non-cirrhotic patients (10). This shift in HE patients' mucosa with higher Enterococcus, Veillonellaceae and Burkholderia abundance may reflect a disease-associated reduction in the normally present autochthonous bacteria that would allow the growth of these potentially pathogenic genera in the mucosa. Patients on rifaximin had worse cognition and inflammation than those with HE without rifaximin, as is usual with clinical practice since rifaximin is initiated in those whose HE is not controlled with lactulose. Therefore, their mucosal microbiome also reflected the worse underlying disease, i.e. a significantly decreased abundance of the autochthonous bacteria. Some of these genera are associated with severe infections in cirrhotic and non-cirrhotic patients (26, 45). Furthermore, these genera were only seen in HE patients' networks and were associated with a higher MELD score, worse endothelial activation, worse cognitive performance (lures, serial dotting and digit symbol tests) and higher systemic inflammation (IL-17) in the HE group. While we found differences in Metastats, there was no clustering noted on PCO or UniFrac. This is not surprising since Metastats is able to detect specific differences between groups at several levels using multiple, random permutations while assessing statistical differences while UniFrac is simply an analysis of phylogenetic distance between taxa, and PCA is an unsupervised clustering, which is not able to incorporate group-specific knowledge or identification of specific features responsible for the differences (22, 34). Most importantly, the abundances found to be different between groups on Metastats have biological plausibility i.e. autochthonous genera were over-represented in the no-HE while pathogenic ones were in the HE group and these were correlated with cognitive, inflammatory and endothelial phenotypes in the direction that was expected.

In the correlation network for patients with HE, a richer and more robust interaction was seen between the microbiome, cognition, inflammation and endothelial activation compared to those without HE. We confirmed the results of prior studies that Alcaligeneceae and Porphyromonadaceae were associated with poor cognitive performance (7, 19). One interesting finding was that the autochthonous bacteria belonging to Lachnospiraceae, Ruminococcaceae and Incertae Sedis XIV had similar beneficial linkages, regardless of the setting. This means that the presence of these bacteria is associated with better cognitive functioning, decreased inflammation and endothelial activation regardless of the early or advanced disease stage. This accords with studies showing that Fecalibacterium and Lachnospiraceae spp are associated with reduced intestinal inflammation in Crohn's disease and extends it into cirrhosis (37, 38). There is also evidence that these bacteria are correlated with markers for reduced inflammation of Th-17 cells in the colon (3). Prior studies have shown that intestinal inflammation can initiate the IL-17/IL-23 system, which is up-regulated in Crohn's disease (9, 12, 20). Correspondingly, we found correlations with markers for the IL-17/IL-23 inflammatory response system in cirrhosis, in both HE and no-HE patients. In our study, IL-17 levels were also correlated with Th1 and Th2 cytokines, such as IL-6 and IL-1b as well as with MELD score. This replicates prior studies which show that IL-1b and IL-6 are essential for converting T-regulatory cells into Th-17 differentiated T cells(1). Also there was a negative correlation between autochthonous bacteria and IL-17 and other inflammatory markers, indicating that the gut-based inflammation may be modulated in the presence of these bacteria. Studies have shown that these autochthonous bacteria support Th-17 polarization and are necessary for maintaining a steady-state of Th17 cells and prevent inflammatory and autoimmune processes (9). This association with peripheral cytokines is interesting because there is evidence linking inflammation and change in T-regulatory cells on brain function in liver disease with or without HE (13, 15, 29, 36). Prior studies have also shown that HE is associated with significantly worse systemic inflammation that can potentially improve with therapy (6, 36). Thus, these inflammatory cytokines are related to or correlated with the mechanism behind the microbiome-associated changes in brain function in HE.

The current study only relied on the presence of bacteria but it is also possible that their end-products, such as the beneficial short-chain fatty acids or the relatively toxic indoles and phenols, may influence clinical outcomes (18, 31). A study of the functional component of the microbes would be important to analyze these effects (18).

We conclude that there is a significant difference in the colonic and stool microbiome in cirrhosis, which persists even when patients are sub-divided into those with and without HE. We also found that the colonic mucosal microbiome of HE patients is significantly different from patients without HE. There is a lower abundance of autochthonous bacterial genera coupled with a higher level of potentially pathogenic bacteria such as Enterooccus and Burkholderia in the HE patients' colonic mucosa. Autochthonous bacteria, Lachnospiraceae, Ruminococcaceae and Incertae Sedis XIV, are associated with better cognition, lower severity of liver disease, decreased inflammation and endothelial activation in both HE and no-HE groups. Autochthonous bacteria, Lachnospiraceae, Ruminococcaceae and Incertae Sedis XIV, are associated with better cognition, lower severity of liver disease, decreased inflammation and endothelial activation in both HE and no-HE groups. However, genera over-represented in the HE patients' mucosa were associated with a pro-inflammatory milieu, higher MELD score and poor cognition. IL-17 was closely linked to IL-6, IL-1b and the potentially pathogenic genera, Enterococcus, Burkholderia and Veillonella only in the HE group. Therefore, the colonic mucosal microbiome of patients with HE is significantly different from patients without HE and is associated with the pro-inflammatory milieu, endothelial activation and poor cognitive performance that is inherent in this patient population.

### REFERENCES for Example 2

1. Afzali et al. Clin Exp Immunol 159: 120-130, 2010.
2. Ahern et al. Immunity 33: 279-288, 2010.
3. Atarashi et al. Science 331: 337-341, 2011.
4. Bajaj et al. Metab Brain Dis, 2012.
5. Bajaj et al. Gastroenterology 135: 1591-1600 e1591, 2008.
6. Bajaj et al. Gastroenterology 140: 478-487 e471, 2011.
7. Bajaj et al. Am J Physiol Gastrointest Liver Physiol 302: G168-175, 2012.
8. Barcenilla et al. Appl Environ Microbiol 66: 1654-1661, 2000.
9. Barnes M J and Powrie F. Science 331: 289-290, 2011.
10. Chen et al. Hepatology 54: 562-572, 2011.
11. Cordoba J. J Hepatol, 2011.
12. D'Elios et al. Expert Opin Ther Targets 14: 759-774, 2010.
13. D'Mello and Swain. Am J Physiol Gastrointest Liver Physiol 301: G749-761, 2011.
14. Folstein et al. J Psychiatr Res 12: 189-198, 1975.
15. Garcia-Martinez and Cordoba. J Hepatol 56: 515-517, 2012.
16. Gillevet et al. Chem Biodivers 7: 1065-1075, 2010.
17. Green et al. J Appl Microbiol 100: 460-469, 2006.
18. Hamer et al. Am J Physiol Gastrointest Liver Physiol 302: G1-9, 2011.
19. Henao-Mejia et al. Nature 482: 179-185, 2012.
20. Huber S and Flavell R A. Immunity 33: 150-152, 2010.
21. Johansson et al. Proc Natl Acad Sci USA 105: 15064-15069, 2008.
22. Jolliffe I T. Principal Component Analysis. New York: Springer-Verlag, 1986.
23. Lane D J. 16s/23s rRNA sequencing. In: Nucleic acid techniques in bacterial systematics, edited by Goodfellow M. West Sussex, England: John Wiley & Sons Ltd, 1991, p. 115-175.
24. Lepage et al. Inflamm Bowel Dis 11: 473-480, 2005.
25. Lozupone C and Knight R. Appl Environ Microbial 71: 8228-8235, 2005.
26. Merli et al. Clin Gastroenterol Hepatol 8: 979-985, 2010.
27. Mutlu et al. Am J Physiol Gastrointest Liver Physiol, 2012.
28. Nava G M and Stappenbeck T S. Gut Microbes 2, 2011.
29. Nguyen et al. J Hepatol, 2011.
30. Ridlon et al. Clin Chim Acta 357: 55-64, 2005.
31. Riggio et al. Am J Gastroenterol 105: 1374-1381, 2010.
32. Romero et al. Med Clin (Bare) 127: 246-249, 2006.
33. Scarpellini et al. Am J Gastroenterol 105: 323-327, 2010.
34. Segata et al. Genome Biol 12: R60, 2011.
35. Shawcross et al. J Hepatol 54: 640-649, 2011.
36. Shawcross et al. Metab Brain Dis 22: 125-138, 2007.
37. Sokol et al. Inflamm Bowel Dis 14: 858-867, 2008.
38. Sokol et al. Proc Natl Acad Sci USA 105: 16731-16736, 2008.
39. Tandon P and Garcia-Tsao G. Semin Liver Dis 28: 26-42, 2008.
40. Teltschik et al. Hepatology, 2011.
41. Weissenbom et al. J Hepatol 34: 768-773, 2001.
42. White et al. PLoS Comput Biol 5: e1000352, 2009.
43. Wiest R and Garcia-Tsao G. Hepatology 41: 422-433, 2005.
44. Wiltfang et al. Metab Brain Dis 14: 239-251, 1999.
45. Wong et al. Gut 54: 718-725, 2005.
46. Zoetendal et al. Appl Environ Microbial 68: 3401-3407, 2002.

While the invention has been described in terms of its preferred embodiments, those skilled in the art will recognize that the invention can be practiced with modification within the spirit and scope of the appended claims. Accordingly, the present invention should not be limited to the embodiments as described above, but should further include all modifications and equivalents thereof within the spirit and scope of the description provided herein.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

We claim:

1. A method of assessing the presence or the risk of development of encephalopathy in a patient with liver disease, comprising the steps of
analyzing gut microflora of said patient in order to determine a gut microbiome signature for said patient;
comparing said gut microbiome signature of said patient to one or more gut microbiome reference signatures, wherein said one or more gut microbiome reference signatures include at least one of a positive gut microbiome reference signature based on results from control subjects with encephalopathy and a negative gut microbiome reference signature based on results from control subjects without encephalopathy; and
if said gut microbiome signature for said patient statistically significantly matches said positive gut microbiome reference signature, then concluding that said patient has or is at risk of developing encephalopathy; and/or
if said gut microbiome signature for said patient statistically significantly matches said negative gut microbiome reference signature, then concluding that said patient does not have or is not at risk of developing encephalopathy.

2. The method of claim 1, wherein a statistically significant match has a P value of 0.05 or less.

3. The method of claim 1, wherein said gut microflora is analyzed in a biological sample selected from a stool sample, a sample of the lumen content, a mucosal biopsy sample, an oral sample, a blood sample and a urine sample.

4. The method of claim 1, wherein said gut microbiome signature includes one or more of: bacterial taxa identified in said gut microflora; bacterial metabolic products in said gut microflora; and proteins in said gut microflora.

5. The method of claim 1, wherein said gut microbiome signature is based on an analysis of amplification products of DNA and/or RNA in said gut microflora.

6. The method of claim 5, wherein said gut microbiome signature is based on an analysis of amplification products of genes coding for one or more of: Small Subunit rRNA, Intervening Transcribed Spacer, and Large Subunit rRNA.

7. The method of claim 5, wherein said gut microbiome signature includes results obtained by assaying the mRNA composition of said biological samples.

8. The method of claim 1, wherein said liver disease is cirrhosis and said encephalopathy is hepatic encephalopathy (HE).

9. The method of claim 1, wherein said gut microbiome signature of said patient includes an indication of the presence and/or relevant abundance of at least one of Alcaligeneceae, Blautia, Burkholderia, Enterobacteriaceae, Fecalibacterium, Fusobacteriaceae, Incertae Sedis XIV, Lachnospiraceae, Porphyromonadaceae, Roseburia , Ruminococcaceae and Veillonellaceae.

10. The method of claim 1, wherein if said gut microflora signature of said patient indicates the presence of Alcaligeneceae and Porphyromonadaceae in said gut microflora, then said concluding step results in a conclusion that said patient has or is at risk of developing encephalopathy.

11. The method of claim 1, further comprising the step of assessing, based on said gut microbiome signature, the presence or the risk of development of inflammation, endotoxemia, and/or endothelial dysfunction in said patient.

12. The method of claim 1, wherein said one or more symptoms of a disease or condition is differentiated from normal conditions using at least one methodology selected from the group consisting of non-parametric multivariate analysis, a Support Vector Machine, correlation network analysis, correlation difference network analysis, Dirichlet models, Bayesian models, and Linear models.

13. A treatment method for a patient with a liver disease, comprising the steps of
analyzing gut microflora of said patient in order to determine a gut microbiome signature for said patient;
comparing said gut microbiome signature of said patient to one or more gut microbiome reference signatures; and, based on said step of comparing,
concluding whether or not said patient has or is at risk for developing at least one of said one or more conditions of interest; and
if said patient has or is at risk for developing at least one of said one or more conditions of interest, then selecting from one or more treatment protocols appropriate for said one or more conditions of interest, and treating the patient according to said one or more treatment protocols selected.

14. The method of claim 13, wherein said one or more conditions of interest include encephalopathy, inflammation, endotoxemia, endothelial dysfunction and coma.

15. The method of claims 13, wherein said one or more treatment protocols include: anti-viral therapy for hepatitis B, C and/or D; weight loss therapy; surgery for non-alcoholic liver disease and obesity-associated liver disease, alcohol abstinence for alcoholic liver disease, therapy for Wilson's disease, alpha-I anti-trypsin repletion, and therapies specific for hepatic encephalopathy and liver transplant.

16. A method of monitoring the efficacy of a treatment protocol in a patient with liver disease or a condition associated with liver disease, comprising the steps of
analyzing gut microflora of said patient in order to determine a gut microbiome signature for said patient;
comparing said gut microbiome signature of said patient to one or more gut microbiome reference signatures, wherein said one or more gut microbiome reference signatures include one or more of a positive gut microbiome reference signature based on results from control subjects with encephalopathy and a negative gut microbiome reference signature based on results from control subjects without encephalopathy; and
if said gut microbiome signature for said patient statistically significantly matches said positive gut microbiome reference signature, then concluding that said treatment protocol is not efficacious; and/or
if said gut microbiome signature for said patient deviates statistically significantly from said negative gut microbiome reference signature, then concluding that said treatment protocol is efficacious,
wherein said analyzing and comparing steps are performed a plurality of times with samples collected from said patient at a plurality of time periods during said treatment protocol.

17. The method of claim 16, wherein said method is carried out prior to commencement of said treatment protocol, during said treatment protocol and/or after cessation of said treatment protocol.
